SEQUENCE LISTING (32 sequences)

SEQ ID NO: 1 - protein, 379 aa, Homo sapiens
MPHSSLHPSIPCPRGHGAQKAALVLLSACLVTLWGLGEPPEHTLRYLVLHLASLQLGLLLNGVCSLAEELRHIHSRYRGSYWRTVRACLGCPLRRGALLLLSIYFYYSLPNAVGPPFTWMLALLGLSQALNILLGLKGLAPAEISAVCEKGNFNVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 2 - protein, 1069 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 3 - protein, 550 aa, Homo sapiens
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 4 - protein, 427 aa, Homo sapiens
MGTPKPRILPWLVSQLDLGQLEGVAWVNKSRTRFRIPWKHGLRQDAQQEDFGIFQAWAEATGAYVPGRDKPDLPTWKRNFRSALNRKEGLRLAEDRSKDPHDPHKIYEFVNSGVGDFSQPDTSPDTNGGGSTSDTQEDILDELLGNMVLAPLPDPGPPSLAVAPEPCPQPLRSPSLDNPTPFPNLGPSENPLKRLLVPGEEWEFEVTAFYRGRQVFQQTISCPEGLRLVGSEVGDRTLPGWPVTLPDPGMSLTDRGVMSYVRHVLSCLGGGLALWRAGQWLWAQRLGHCHTYWAVSEELLPNSGHGPDGEVPKDKEGGVFDLGPFIVDLITFTEGSGRSPRYALWFCVGESWPQDQPWTKRLVMVKVVPTCLRALVEMARVGGASSLENTVDLHISNSHPLSLTSDQYKAYLQDLVEGMDFQGPGES

SEQ ID NO: 5 - DNA, 3505 bp, Homo sapiens
agtttcactttcccttttgtaacgtcagctgaagggaaacaaacaaaaaggaaccagaggccacttgtatatataggtctcttcagcatttattggtggcagaagaggaagatttctgaagagtgcagctgcctgaaccgagccctgccgaacagctgagaattgcactgcaaccatgagtgagaacaataagaattccttggagagcagcctacggcaactaaaatgccatttcacctggaacttgatggagggagaaaactccttggatgattttgaagacaaagtattttaccggactgagtttcagaatcgtgaattcaaagccacaatgtgcaacctactggcctatctaaagcacctcaaagggcaaaacgaggcagccctggaatgcttacgtaaagctgaagagttaatccagcaagagcatgctgaccaggcagaaatcagaagtctggtcacctggggaaactatgcctgggtctactatcacatgggccgactctcagacgttcagatttatgtagacaaggtgaaacatgtctgtgagaagttttccagtccctatagaattgagagtccagagcttgactgtgaggaagggtggacacggttaaagtgtggaggaaaccaaaatgaaagagcgaaggtgtgctttgagaaggctctggaaaagaagccaaagaacccagaattcacctctggactggcaatagcaagctaccgtctggacaactggccaccatctcagaacgccattgaccctctgaggcaagccattcggctgaatcctgacaaccagtaccttaaagtcctcctggctctgaagcttcataagatgcgtgaagaaggtgaagaggaaggtgaaggagagaagttagttgaagaagccttggagaaagccccaggtgtaacagatgttcttcgcagtgcagccaagttttatcgaagaaaagatgagccagacaaagcgattgaactgcttaaaaaggctttagaatacataccaaacaatgcctacctgcattgccaaattgggtgctgctatagggcaaaagtcttccaagtaatgaatctaagagagaatggaatgtatgggaaaagaaagttactggaactaataggacacgctgtggctcatctgaagaaagctgatgaggccaatgataatctcttccgtgtctgttccattcttgccagcctccatgctctagcagatcagtatgaagacgcagagtattacttccaaaaggaattcagtaaagagcttactcctgtagcgaaacaactgctccatctgcggtatggcaactttcagctgtaccaaatgaagtgtgaagacaaggccatccaccactttatagagggtgtaaaaataaaccagaaatcaagggagaaagaaaagatgaaagacaaactgcaaaaaattgccaaaatgcgactttctaaaaatggagcagattctgaggctttgcatgtcttggcattccttcaggagctgaatgaaaaaatgcaacaagcagatgaagactctgagaggggtttggagtctggaagcctcatcccttcagcatcaagctggaatggggaatgaagaatagagatgtggtgcccactaggctactgctgaaagggagctgaaattcctccaccaagttggtattcaaaatatgtaatgactggtatggcaaaagattggactaagacactggccataccactggacagggttatgttaacacctgaattgctgggtcttgagagagcccaaggagttctgggagagggaccagattggggggtaggtccacgggcttggtgatagaattatttctcgattgacttcttgagtgcaatttgaactgtaacatttgcttagtcacctttagtggagtaatctactgggcttgtttctatatttatataaagcagccaaatccttcatgtaatattgaagtccatttttgcaatgttgttccatacttggagtcattttgcatcccatagaggttagtcctgcatagccagtaatgtgctaagttcatccaaaagctggcggaccaaagtctaaatagggctcagtatcccccatcgcttatctctgcctccttcctcctccttcccagtctatcatcaaccttgagtattctacacaatgtgaattcaagtgcctgattaattgaggtggcaacatagtttgagacgagggcagagaacaggaagatacatagctagaagcgacgggtacaaaaagcaatgtgtacaagaagactttcagcaagtatacagagagttcacctctactctgccctcctcatagtcataatgtagcaagtaaagaatgagaatggattctgtacaatacactagaaaccaacataatgtatttctttaaaacctgtgtgaaaaaataaatgttccaccagtagggataggggaaaagtaaccaaaagagagaaagagaaaggaatgctggtttatctttgtagattgtaatcgaatggagaaatttgcagtattttagccactattaggaattttttttttttgtaaaatgaagactgaactctgttcaaatgctttcatgaacctggtttgagacggtaggaaagcaacaaaacgtgggaacctggtgactaagggcctggtgcaaggacttgggaaatgtcattgataatagatggtggggttttcccccctttagaaatgttggatattaagtgatataaacacttcttttaactccgaaaatcttctgagaaatcacaaaattcacggtatgcttggaacgattgagattttctaggtagatgctgaatagcctagacatcaaagttggtgtgaaccaaaatagagtcagctgacccagcatcagccacactctgggttggaaaatgtttgcctgttggaattaatttaagcttaagtatatatcaacattattttattgtgcaattaaaacaatacaaattcatggttttttaaagttaaaaattctaaccactgtaacaacagtttttgtgttattttctgtattaaacatcttgttgcacgcatttgaggtcatcagggtgcaaaatttgtattcctgaaaatgtcatatattttcattaataaataacctaaatatgataaaacataaagcagtgttctggttcatctggaattttgctgtactttaaatctttcagactcagctactgataaatgaaacgttacacaggtgtgaaccaaatccaaataacctcgactggtctactatcataatcacctgaacagaacaaaactttttcctcagctttaagagtccagggcttcggataacagctgccatctgccacctgctaccattgacctacgtgaacacagacattctgtctccaccttgatggtgggtgggctgctccccttttctttgttaaattttgtgctttcatcacattttctctattctgacctctgttatgagaaataaaagtcactgattccattttaaaaaaaaaaa

SEQ ID NO: 6 - DNA, 4396 bp, Homo sapiens
gcaaggacacacccacagcttacaccattggctgctgtttagctcccttatataacactgtcttggggtttaaacgtaactgaaaatccacaagacagaatagccagatctcagaggagcctggctaagcaaaaccctgcagaacggctgcctaatttacagcaaccatgagtacaaatggtgatgatcatcaggtcaaggatagtctggagcaattgagatgtcactttacatgggagttatccattgatgacgatgaaatgcctgatttagaaaacagagtcttggatcagattgaattcctagacaccaaatacagtgtgggaatacacaacctactagcctatgtgaaacacctgaaaggccagaatgaggaagccctgaagagcttaaaagaagctgaaaacttaatgcaggaagaacatgacaaccaagcaaatgtgaggagtctggtgacctggggcaactttgcctggatgtattaccacatgggcagactggcagaagcccagacttacctggacaaggtggagaacatttgcaagaagctttcaaatcccttccgctatagaatggagtgtccagaaatagactgtgaggaaggatgggccttgctgaagtgtggaggaaaaaattatgaacgggccaaggcctgctttgaaaaggtgcttgaagtggaccctgaaaaccctgaatccagcgctgggtatgcgatctctgcctatcgcctggatggctttaaattagccacaaaaaatcacaagccattttctttgcttcccctaaggcaggctgtccgcttaaatccagacaatggatatattaaggttctccttgccctgaagcttcaggatgaaggacaggaagctgaaggagaaaagtacattgaagaagctctagccaacatgtcctcacagacctatgtctttcgatatgcagccaagttttaccgaagaaaaggctctgtggataaagctcttgagttattaaaaaaggccttgcaggaaacacccacttctgtcttactgcatcaccagatagggctttgctacaaggcacaaatgatccaaatcaaggaggctacaaaagggcagcctagagggcagaacagagaaaagctagacaaaatgataagatcagccatatttcattttgaatctgcagtggaaaaaaagcccacatttgaggtggctcatctagacctggcaagaatgtatatagaagcaggcaatcacagaaaagctgaagagaattttcaaaaattgttatgcatgaaaccagtggtagaagaaacaatgcaagacatacatttccactatggtcggtttcaggaatttcaaaagaaatctgacgtcaatgcaattatccattatttaaaagctataaaaatagaacaggcatcattaacaagggataaaagtatcaattctttgaagaaattggttttaaggaaacttcggagaaaggcattagatctggaaagcttgagcctccttgggttcgtctacaaattggaaggaaatatgaatgaagccctggagtactatgagcgggccctgagactggctgctgactttgagaactctgtgagacaaggtccttaggcacccagatatcagccactttcacatttcatttcattttatgctaacatttactaatcatcttttctgcttactgttttcagaaacattataattcactgtaatgatgtaattcttgaataataaatctgacaaaatattagttgtgttcaacaattagtgaaacagaatgtgtgtatgcatgtaagaaagagaaatcatttgtatgagtgctatgtagtagagaaaaaatgttagttaactttgtaggaaataaaacattggacttacactaaatgtttaattcattcattttattgtgaaataaaaataaaatccttagctcctccaccaactgaacagaccctcttggccaaggagaccccagaaaccttaaaaactaagtttcccaaccatgacaagatgagagatcattcacacctcattatattccctcccttgctaactgccattggactttttccactgagttaaacagaaacccatggaaaacaaagaacagaagactcactccttggctgacttcacctagctcactccacgtagcgccacagccagactcccctcccctcttgcggtttccacatgacaactgatcagccttccctcctgataagtgaccactgcccacagactggttctggccagtccatggaggctgcacacagggtgcctctatgtcctttgtttcaccttttgatatagaaaggctaattttgctgtattttaatgttaagtctccaccacagagtgaacacagaatgcatgtgacatacatgtttacataccactattgtgtgactgcccctcatgaatattcatagccccccataacctgttaactatgtgtgtctagccaatccaccaaccataaaacttctgtaataccctcccttcctccaagagcctgcttttggttgctgtggtaggctctgcttcccaggctgcaggttgcaggagaggaggctgcagtggctcacgcctgtaatctcagcacttcgatgggacgaggcaggcagatcacctgaacccaggagttcgagagcagccttggcaatggcaaaaccaaccgtctctacaaaaaatgcaaaaacttagctgggtgtggtggcatgcacctgtagcttcagttccagctactcaggaggctgaggtgagtggactgctggagccagggagttcgaggctgcagtgtcgagatcttgccactgcactccattctggatgatagaacgagaccccatctcaaaaaaaaaaaaagttctctccaattgtatatagcttgtgattttatgtcaacactatcaataaatagctttcagtgcaagaaaccaaaaatactgtaataaacaggcacatattcttcccaaacctcatgcagtttacaatctagtgagagacacagatagcagtacagagtcaattaaaggttagttttcttcatgaagatgttttaattttaattcaatgtgaaagggttccaaggagtttatcttgttttatgccattttatttgaagcactacttactaagtcatttgctgatattaatctagttaaatcaagaaatattacatgaaaatgttgctaaatcagagatcatgggtaacaatcacctttgattatgaataatcatattttattgaaaggcaaggcacaacaaataataagaaggaaaaaataaataagcaatgttattgatctttcattctgtatatgttttggggggaatatactagtttcttttagtggctgtaacaaattaccacaaacttggtgacttaaaatttcacagatttactctttcttacagttctggaggtcagaagtctgaaatgggtttcaatgagccaaagtcaaggtattgatgacgctacactcctccggaggctctaggcagatagccttttccagcttccagaggctgcctgaattctttcatccatcttaaaaaccaacagtgtagtagcctcaaatctctctctctgcttccttcttcacatctccttctctcctctgactcttttgcctctttcttctaaggacgcaccaggtccacctgcataatccagaataattgccccatccgcaaatccttaatttaataacatctgcaaagtcccttttgctatgtaaagtagcatgttcacaggttctggagacttggccatggatacgattgcggggggggcattattcttaccacagagcaccccaagaaaatctccaaattttgggcttccaatccattttgcttcaattatttaatatttttactccttccagtagatactgatttcatccattgcccttaagaaggtaggacagagattatggcacatctcacattaaatgctatattttcgttggaaatacattttttgcttcaacttttattttaaattcaagggtacatgtgcaggatgttcaggtttgttacacaggtaaacgtgtgccatggcggtttgctgaacagatcatcccatcaccaacagatcatcccattgagaggtgaagccggctgggcttctgggttgggtggggacttggagaacttttctgtctagctaaagtattgtaaaatggaccagtcaacactctgtaaaatggaccaatcagctctctgtaaaatggaccaatcagcaggatgtgggtggggccaagtaagggaataaaagcaggccacccgagctggcagcggcaacccgctcgggtccccttccatgctgtggaagttttgttctttcgctctttcaataaatcttgctgctgctcaaaaaaaaaaaaaaaaaa

SEQ ID NO: 7 - DNA, 685 bp, Homo sapiens
ataatagggccggtgctgcctgccgaagccggcggctgagaggcagcgaactcatctttgccagtacaggagcttgtgccgtggcccacagcccacagcccacagccatgggctgggacctgacggtgaagatgctggcgggcaacgaattccaggtgtccctgagcagctccatgtcggtgtcagagctgaaggcgcagatcacccagaagatcggcgtgcacgccttccagcagcgtctggctgtccacccgagcggtgtggcgctgcaggacagggtcccccttgccagccagggcctgggccccggcagcacggtcctgctggtggtggacaaatgcgacgaacctctgagcatcctggtgaggaataacaagggccgcagcagcacctacgaggtacggctgacgcagaccgtggcccacctgaagcagcaagtgagcgggctggagggtgtgcaggacgacctgttctggctgaccttcgaggggaagcccctggaggaccagctcccgctgggggagtacggcctcaagcccctgagcaccgtgttcatgaatctgcgcctgcggggaggcggcacagagcctggcgggcggagctaagggcctccaccagcatccgagcaggatcaagggccggaaataaaggctgttgtaaagagaaaaaaaaaaaaaaaaaaaaa

SEQ ID NO: 8 - DNA, 2853 bp, Homo sapiens
gctctgctccaggcatctgccacaatgtgggtgcttacacctgctgcttttgctgggaagctcttgagtgtgttcaggcaacctctgagctctctgtggaggagcctggtcccgctgttctgctggctgagggcaaccttctggctgctagctaccaagaggagaaagcagcagctggtcctgagagggccagatgagaccaaagaggaggaagaggaccctcctctgcccaccaccccaaccagcgtcaactatcacttcactcgccagtgcaactacaaatgcggcttctgtttccacacagccaaaacatcctttgtgctgccccttgaggaagcaaagagaggattgcttttgcttaaggaagctggtatggagaagatcaacttttcaggtggagagccatttcttcaagaccggggagaatacctgggcaagttggtgaggttctgcaaagtagagttgcggctgcccagcgtgagcatcgtgagcaatggaagcctgatccgggagaggtggttccagaattatggtgagtatttggacattctcgctatctcctgtgacagctttgacgaggaagtcaatgtccttattggccgtggccaaggaaagaagaaccatgtggaaaaccttcaaaagctgaggaggtggtgtagggattatagagtcgctttcaagataaattctgtcattaatcgtttcaacgtggaagaggacatgacggaacagatcaaagcactaaaccctgtccgctggaaagtgttccagtgcctcttaattgagggtgagaattgtggagaagatgctctaagagaagcagaaagatttgttattggtgatgaagaatttgaaagattcttggagcgccacaaagaagtgtcctgcttggtgcctgaatctaaccagaagatgaaagactcctaccttattctggatgaatatatgcgctttctgaactgtagaaagggacggaaggacccttccaagtccatcctggatgttggtgtagaagaagctataaaattcagtggatttgatgaaaagatgtttctgaagcgaggaggaaaatacatatggagtaaggctgatctgaagctggattggtagagcggaaagtggaacgagacttcaacacaccagtgggaaaactcctagagtaactgccattgtctgcaatactatcccgttggtatttcccagtggctgaaaacctgattttctgctgcacgtggcatctgattacctgtggtcactgaacacacgaataacttggatagcaaatcctgagacaatggaaaaccattaactttacttcattggcttataaccttgttgttattgaaacagcacttctgtttttgagtttgttttagctaaaaagaaggaatacacacaggaataatgaccccaaaaatgcttagataaggcccctatacacaggacctgacatttagctcaatgatgcgtttgtaagaaataagctctagtgatatctgtgggggcaaaatttaatttggatttgattttttaaaacaatgtttactgcgatttctatatttccattttgaaactatttcttgttccaggtttgttcatttgacagagtcagtattttttgccaaatatccagataaccagttttcacatctgagacattacaaagtatctgcctcaattatttctgctggttataatgcttttttttttttgcctttatgccattgcagtcttgtactttttactgtgatgtacagaaatagtcaacagatgtttccaagaacatatgatatgataatcctaccaattttcaagaagtctctagaaagagataacacatggaaagacggcgtggtgcagcccagcccacggtgcctgttccatgaatgctggctacctatgtgtgtggtacctgttgtgtccctttctcttcaaagatccctgagcaaaacaaagatacgctttccatttgatgatggagttgacatggaggcagtgcttgcattgctttgttcgcctatcatctggccacatgaggctgtcaagcaaaagaataggagtgtagttgagtagctggttggccctacatttctgagaagtgacgttacactgggttggcataagatatcctaaaatcacgctggaaccttgggcaaggaagaatgtgagcaagagtagagagagtgcctggatttcatgtcagtgaagccatgtcaccatatcatatttttgaatgaactctgagtcagttgaaatagggtaccatctaggtcagtttaagaagagtcagctcagagaaagcaagcataagggaaaatgtcacgtaaactagatcagggaacaaaatcctctccttgtggaaatatcccatgcagtttgttgatacaacttagtatcttattgcctaaaaaaaaatttcttatcattgtttcaaaaaagcaaaatcatggaaaatttttgttgtccaggcaaataaaaggtcattttaatttaaaaaaaaaaaaaaaaaaaaaaaaaaaaaaaaggccaaggaaaaaaaatattcctacttaaattttaagtctataattcaatttaaatatgtgtgtgtctcatccaggataggataggttgtcttctattttccattttacctatttactttttttgtaagaaaagagaagaatgaattctaaagatgttccccatgggttttgattgtgtctaagctatgatgaccttcatataatcagcataaacataaaacaaattttttacttaacatgagtgcactttactaatcctcatggcacagtggctcacgcctgtaatcccagcacttggggaggacaatgtggggtggatcacgaggtc

SEQ ID NO: 9 - protein, 317 aa, Homo sapiens
MRRASRDYTKYLRGSEEMGGGPGAPHEGPLHAPPPPAPHQPPAASRSMFVALLGLGLGQVVCSVALFFYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 10 - DNA, 1605 bp, Homo sapiens
gaagaaaccaccggaaggaaccatctcactgtgtgtaaacatgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaagagagctctgtctggaccccaaggaaaactgggtgcagagggttgtggagaagtttttgaagagggctgagaattcataaaaaaattcattctctgtggtatccaagaatcagtgaagatgccagtgaaacttcaagcaaatctacttcaacacttcatgtattgtgtgggtctgttgtagggttgccagatgcaatacaagattcctggttaaatttgaatttcagtaaacaatgaatagtttttcattgtaccatgaaatatccagaacatacttatatgtaaagtattatttatttgaatctacaaaaaacaacaaataatttttaaatataaggattttcctagatattgcacgggagaatatacaaatagcaaaattgaggccaagggccaagagaatatccgaactttaatttcaggaattgaatgggtttgctagaatgtgatatttgaagcatcacataaaaatgatgggacaataaattttgccataaagtcaaatttagctggaaatcctggatttttttctgttaaatctggcaaccctagtctgctagccaggatccacaagtccttgttccactgtgccttggtttctcctttatttctaagtggaaaaagtattagccaccatcttacctcacagtgatgttgtgaggacatgtggaagcactttaagttttttcatcataacataaattattttcaagtgtaacttattaacctatttattatttatgtatttatttaagcatcaaatatttgtgcaagaatttggaaaaatagaagatgaatcattgattgaatagttataaagatgttatagtaaatttattttattttagatattaaatgatgttttattagataaatttcaatcagggtttttagattaaacaaacaaacaattgggtacccagttaaattttcatttcagataaacaacaaataattttttagtataagtacattattgtttatctgaaattttaattgaactaacaatcctagtttgatactcccagtcttgtcattgccagctgtgttggtagtgctgtgttgaattacggaataatgagttagaactattaaaacagccaaaactccacagtcaatattagtaatttcttgctggttgaaacttgtttattatgtacaaatagattcttataatattatttaaatgactgcatttttaaatacaaggctttatatttttaactttaagatgtttttatgtgctctccaaattttttttactgtttctgattgtatggaaatataaaagtaaatatgaaacatttaaaatataatttgttgtcaaagtaaaaaaaaaaaaaaa

SEQ ID NO: 11 - DNA, 1498 bp, Homo sapiens
accaaacctcttcgaggcacaaggcacaacaggctgctctgggattctcttcagccaatcttcattgctcaagtgtctgaagcagccatggcagaagtacctgagctcgccagtgaaatgatggcttattacagtggcaatgaggatgacttgttctttgaagctgatggccctaaacagatgaagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaaagagagctgtacccagagagtcctgtgctgaatgtggactcaatccctagggctggcagaaagggaacagaaaggtttttgagtacggctatagcctggactttcctgttgtctacaccaatgcccaactgcctgccttagggtagtgctaagaggatctcctgtccatcagccaggacagtcagctctctcctttcagggccaatccccagcccttttgttgagccaggcctctctcacctctcctactcacttaaagcccgcctgacagaaaccacggccacatttggttctaagaaaccctctgtcattcgctcccacattctgatgagcaaccgcttccctatttatttatttatttgtttgtttgttttattcattggtctaatttattcaaagggggcaagaagtagcagtgtctgtaaaagagcctagtttttaatagctatggaatcaattcaatttggactggtgtgctctctttaaatcaagtcctttaattaagactgaaaatatataagctcagattatttaaatgggaatatttataaatgagcaaatatcatactgttcaatggttctgaaataaacttcactgaag

SEQ ID NO: 12 - DNA, 225 bp, Homo sapiens
gcatcacttgctgctgacacgccgaccgcctgctgcttcagctacacctcccggcagattccacagaatttcatagctgactactttgagacgagcagccagtgctccaagcccggtgtcatcttcctaaccaagcgaagccggcaggtctgtgctgaccccagtgaggagtgggtccagaaatatgtcagcgacctggagctgagtgcctgaggggtccagaag

SEQ ID NO: 13 - DNA, 19 bp, Artificial Sequence
  PCR primer
acttcagttcagaatggag

SEQ ID NO: 14 - DNA, 18 bp, Artificial Sequence
  oliognucleotide primer
tattctgtgaaggcgtcc

SEQ ID NO: 15 - DNA, 20 bp, Artificial Sequence
  oligonucleotide primer
cagcgccccaccaagctcaa

SEQ ID NO: 16 - DNA, 20 bp, Artificial Sequence
  oligonucleotide primer
tgctccctcgctcccaagca

SEQ ID NO: 17 - DNA, 18 bp, Artificial Sequence
  oligonucleotide
gacttccaagctggccgt

SEQ ID NO: 18 - DNA, 19 bp, Artificial Sequence
  oligonucleotide primer
gaattctcagccctcttca

SEQ ID NO: 19 - DNA, 23 bp, Artificial Sequence
  oligonucleotide primer
aacaggctgctctgggattctct

SEQ ID NO: 20 - DNA, 24 bp, Artificial Sequence
  oligonucleotide
tgaagggaaagaaggtgctcaggt

SEQ ID NO: 21 - DNA, 20 bp, Artificial Sequence
  oligonucleotide primer
gctgcccttgctgtcctcct

SEQ ID NO: 22 - DNA, 20 bp, Artificial Sequence
  oligonucleotide primer
ggtcagcacagacctgccgg

SEQ ID NO: 23 - DNA, 20 bp, Artificial Sequence
  oligonucleotide primer
tccagcccctacaggattga

SEQ ID NO: 24 - DNA, 20 bp, Artificial Sequence
  oligonucleotide
ttcgggtccttttccagagc

SEQ ID NO: 25 - DNA, 21 bp, Artificial Sequence
  oligonucleotide primer
ctggagcagctgaatggaaag

SEQ ID NO: 26 - DNA, 22 bp, Artificial Sequence
  oligonucleotide primer
cttctccgtcatctccataggg

SEQ ID NO: 27 - DNA, 20 bp, Artificial Sequence
  oligonucleotide primer
agcaggtgtgtgcctatcac

SEQ ID NO: 28 - DNA, 20 bp, Artificial Sequence
  oligonucleotide primer
tcagccagcagaacaggatg

SEQ ID NO: 29 - DNA, 20 bp, Artificial Sequence
  oligonucleotide primer
gaggtgacagccttctaccg

SEQ ID NO: 30 - DNA, 20 bp, Artificial Sequence
  oligonucleotide primer
agtacttcattgcggcactg

SEQ ID NO: 31 - DNA, 20 bp, Artificial Sequence
  oligonucleotide primer
agaacacgagaccaatggtg

SEQ ID NO: 32 - DNA, 20 bp, Artificial Sequence
  oligonucleotide
cccgtgtcccaggggtcacg

(19) United States
(12) Patent Application Publication
(10) Pub. No.: US 2017/0146519 A1
(43) Pub. Date: May 25, 2017

(54) STING AGONISTS AND METHODS OF SELECTING STING AGONISTS

(71) Applicants: Victor DeFilippis, Tigard, OR (US); Tina Sali, Gladstone, OR (US); Kara Pryke, Beaverton, OR (US); Jinu Abraham, Beaverton, OR (US); Andrew Liu, Beaverton, OR (US); Iris Archer, Beaverton, OR (US); Kayla Sheridan, Beaverton, OR (US); Aaron Nilsen, Portland, OR (US); Rebecca Broeckel, Beaverton, OR (US); Jessica Smith, Portland, OR (US); Lisi Amsler, Portland, OR (US); Daniel Streblow, Beaverton, OR (US); Andrew Placzek, Portland, OR (US)

(72) Inventors: Victor DeFilippis, Tigard, OR (US); Tina Sali, Gladstone, OR (US); Kara Pryke, Beaverton, OR (US); Jinu Abraham, Beaverton, OR (US); Andrew Liu, Beaverton, OR (US); Iris Archer, Beaverton, OR (US); Kayla Sheridan, Beaverton, OR (US); Aaron Nilsen, Portland, OR (US); Rebecca Broeckel, Beaverton, OR (US); Jessica Smith, Portland, OR (US); Lisi Amsler, Portland, OR (US); Daniel Streblow, Beaverton, OR (US); Andrew Placzek, Portland, OR (US)

(73) Assignee: OREGON HEALTH & SCIENCE UNIVERSITY, PORTLAND, OR (US)

(21) Appl. No.: 15/357,685

(22) Filed: Nov. 21, 2016

Related U.S. Application Data
(60) Provisional application No. 62/258,339, filed on Nov. 20, 2015.

(51) Int. Cl. G01N 33/50; C07D 279/16; C07D 417/12

(52) U.S. Cl. CPC G01N 33/5041; C07D 417/12; C07D 279/16; G01N 2333/705

(57) ABSTRACT

Disclosed are small molecules capable of activating the type I interferon (IFN) response by way of the transcription factor IFN regulatory factor 3 (IRF3) were identified. A high throughput in vitro screen yielded 4-(2-chloro-6-fluorobenzyl)-N-(furan-2-ylmethyl)-3-oxo-3,4-dihydro-2H-benzo[b][1,4]thiazine-6-carboxamide (referred to herein as G10), which was found to trigger IRF3/IFN-associated transcription in human fibroblasts. To define cellular proteins essential to elicitation of the antiviral activity by the compound a reverse genetics approach that utilized genome editing via CRISPR/Cas9 technology was employed. This allowed the identification of IRF3, the IRF3-activating adaptor molecule STING, and the IFN-associated transcription factor STAT1 as required for observed gene induction and antiviral effects.

### RELATED APPLICATIONS

This application claims the benefit of U.S. Provisional Patent Application 62/258,339, entitled STING AGONISTS, filed on 20 Nov. 2015, and which is incorporated by reference herein.

### ACKNOWLEDGEMENT OF GOVERNMENT SUPPORT

Work leading to this invention was supported by the United States government under the terms of grant numbers U54 AI081680, U19 AI 109680, and HHSN272201400055C, all of which were awarded by the National Institutes of Health. The United States government has certain rights to this invention.

### BACKGROUND

The innate immune system includes an array of sentinel proteins termed pattern recognition receptors (PRRs) that sense and react to microbe- and danger-associated molecular patterns (reviewed in Broz P et al, Nat Rev Immunol 13, 551-565 (2013); incorporated by reference herein). These patterns are often constituents or replication intermediates of intracellular (especially viral) pathogens. PRRs respond to this engagement by initiating signaling pathways that bring about the expression or processing of cytokines, chemokines, and effector molecules that both directly block microbial replication and facilitate related adaptive immune processes. As such, PRRs represent an essential first line of immunological defense against infection and are the target of both microbial inhibitory phenotypes as well as pharmacologic manipulation for therapeutic purposes (reviewed in Es-Saad S et al, Curr Opin Virol 2, 622-628 (2012); incorporated by reference herein).

Synthesis and secretion of interferon (IFN) proteins is often a primary outcome of PRR mediated signaling. This includes multiple subtypes of IFNα and β (type I IFN) as well as IFN λ1-3 (type III IFN). IFNs act via cognate cell surface receptors by triggering a phosphorylation cascade involving Janus and tyrosine kinases (Jak1, Tyk2) and signal transducer and activator of transcription 1 and 2 (STAT1/2) transcription factors that amplify the expression of antiviral effector and other immune stimulatory genes conventionally termed IFN-stimulated genes (ISGs). PRR-mediated expression of IFNβ is particularly well characterized and requires phosphorylation of the transcription factor IFN regulatory factor 3 (IRF3) by serine kinases TANK Binding kinase 1 (TBK1) and I Kappa B kinase ε (IKKε) (Sharma S et al, Science 300, 1148-1151 (2003); incorporated by reference herein). This occurs primarily through pathways that utilize specific adaptor proteins acting as integration points for upstream PRRs. TIR-domain-containing adaptor-inducing IFNβ (TRIF; also called TICAM1) is required for signals initiated by Toll-like receptors (TLRs) 3 and 4 (Yamamoto M et al, Science 301, 640-643 (2003) and Oshiumi H et al, Nat Immunol 4, 161-167 (2003); both of which are incorporated by reference herein). IFN promoter stimulator 1 (IPS-1; also called MAVS, VISA, or Cardif) is employed by RIG-I and MDA5, that both sense cytoplasmic dsRNA (Xu L G et al, Mol Cell 19, 727-740 (2005); Kawai T et al, Nat Immunol 6, 981-988 (2005); Seth R B et al, Cell 122, 669-682 (2005); Meylan E et al, Nature 437, 1167-1172 (2005); all of which are incorporated by reference herein. Stimulator of IFN genes (STING; also called MITA, TMEM173, MPYS, ERIS) (Ichikawa H et al, Nature 455, 674-678 (2008); Zhong B et al, 29, 538-550 (2008); and Sun W et al, Proc Natl Acad Sci USA 106, 8653-8658 (2009); all of which are incorporated by reference herein) is actually both a PRR for cyclic dinucleotides (CDN) via a binding pocket in its C-terminal cytoplasmic domain (CTD) Burdette D L et al, Nature 478, 515-518 (2012); Sun L et al, Science 339, 786-791 (2013); and Wu J et al, Science 339, 826-830 (2013); all of which are incorporated by reference herein) as well as an adaptor molecule for multiple cytoplasmic receptors of dsDNA (Unterholzner L et al, Nat Immunol 11, 1004 (2010); Stavrou S et al, Cell Host and Microbe 17, 478-488 (2015); and DeFilippis V R et al, J Virol 84, 585-598 (2010); all of which are incorporated by reference herein). Given the importance of these pathways for innate immune activation and antimicrobial protection they have been the focus of broad and intense research aimed at both understanding their physiological effects and harnessing their potential for contributions to immune-based therapeutics.

Given the ability of the IFN system to render cells and tissues refractory to replication of a wide array of virus types as well as its role in coordinating adaptive immune responses, pharmacologic IFN stimulation has been suggested as a broad spectrum antiviral strategy (Ireton R C et al, Antiviral Res 108, 156-164 (2014); Patel D A et al, PLoS ONE doi:10.1371 (2012); Wong J P et al, Vaccine 27, 3481-3483 (2009); Silin D S et al, Curr Pharm Des 15, 1238-1247 (2009); all of which are incorporated by reference herein). Moreover, factors capable of yielding therapeutic effects via activation of IRF3-mediated responses have been identified and biologically validated. This includes agonists of TLRs shown to block replication of some chronic viruses (Wu J et al, Hepatolory 46, 1769-1778 (2007); Isogawa M et al, J Virol 79, 7269-7272 (2005); and Svensson A et al, J Reprod Immunol 74, 114-123 (2007); all of which are incorporated by reference herein) as well as enhance vaccine immunity (reviewed in (Maisonneuve C et al, Proc Natal Acad Sci USA 111, 12294-12299 (2014); incorporated by reference herein). Similarly, stimulation of the RIG-I/MDA5/IPS-1 by synthetic nucleic acids can be employed for antiviral outcomes against diverse acute viruses (Goulet M L et al, PLoS Pathol 9, e1003298 (2013) and Olagnier D et al, J Virol 88, 4180-4194 (2014); both of which are incorporated by reference herein). Intriguingly, two synthetic small molecules, 10-carboxymethyl-9-acridanone (CMA) (Kramer M J et al, Antimicrobial Agents and Chemotherapy 9, 233-238 (1976); incorporated by reference herein) and the chemically unrelated 5,6-dimethylxanthenone-4-acetic acid (DMXAA) (Perera P Y et al, J Immunol 153, 4684-4693 (1994); incorporated by reference herein) are each capable of activating the STING pathway. Both molecules block multiple, even drug-resistant viruses (Guo F et al, Antimicrob Agents Chemother doi:10.1128/AAC.04321-14 (2014); Cheng G et al, Am J Respir Cell Mol Biol 45, 480-488 (2011); Shirey K A et al, J Leukoc Biol 89, 351-357 (2011); all of which are incorporated by reference herein). Intriguingly, DMXAA exhibits other immunotherapeutic effects including vaccine adjuvanticity (Tang C K et al, PLoS ONE 8, e60038 (2013); Blaauboer S M et al, J Immunol 192, 492-502 (2014); both of which are incorporated by reference herein), anti-angiogenic vascular disruption promoting tumor necrosis (Wallace A et al, Cancer Res 67, 7011-7019 (2007) and Jassar A S, Cancer Res 65, 11752-11761 (2005); incorporated by reference herein), and immune-mediated clearance of solid tumors (Corrales L et al, Cell Rep 11, 1018-1030 (2015); incorporated by reference herein). Unfortunately, CMA and DMXAA were found to only function in mouse, not human cells and tissues (Caviar T et al, EMBO J 32, 1440-1450 (2013); Kim S et al, ACS Chem Biol 8, 1396-1401 (2013); and Kim S et al, ACS Chem Biol 8, 1396-1401 (2013); all of which are incorporated by reference herein and thus were not effective in clinical trials. While analogs of cross-specific stimulatory CDNs have been synthesized (Conlon J et al, J Immunol 190, 5216-5225 (2013); incorporated by reference herein), to our knowledge there exists no published biological characterization of novel synthetic molecular entities that activate human STING dependent innate responses, despite the high and multi-pronged therapeutic potential of exploiting this important immunological protein.

Members of the Alphavirus genus include mosquito-transmitted agents that are re-emerging worldwide and can lead to significant morbidity and mortality (reviewed in (Weaver S C et al, Antiviral Res 94, 242-257 (2012); incorporated by reference herein). Among these is Chikungunya virus (CHIKV), which, despite its evolutionary origin in the Old World, is currently experiencing a severe outbreak in the Caribbean, Central, and South America. Since it first arrived in the Western hemisphere in December 2013 over one million suspected and confirmed cases are estimated to have occurred (Johansson M A, Trends Parasitol 31, 43-45 (2015); incorporated by reference herein). CHIKV disease is characterized by severe joint pain that can persist for months to years. Venezuelan Encephalitis virus (VEEV) is a related virus belonging to the New World clade that has experienced numerous outbreaks in South and Central America as well as southern Texas (Zehmer R G et al, Health Serv Rep 89, 278-282 (1974); incorporated by reference herein). VEEV is a much more deadly agent with fatality rates at approximately 20% but that can reach up to 35% in children (reviewed in (Go Y Y et al, Clin Exp Vaccine Res 3, 58-77 (2014); incorporated by reference herein). Currently no FDA-approved antiviral drugs or vaccines exist for either virus. Interestingly, however, both viruses are extremely sensitive to type I IFN (Couderc T et al, PLoS Pathogens, 10.1371/journal.ppat.0040029 (2008); Lukaszewski R A and Brooks T J, J Virol 74, 5006-5015 (2000); Pinto A J et al, J Interferon Res 10, 293-298 (1990); all of which are incorporated by reference herein). Moreover, being RNA-based viruses their infection triggers IRF3/IFN activation via the IPS-1 pathway (White L K et al, J Virol 85, 606-620 (2011); incorporated by reference herein) and as such may not exhibit evasion phenotypes directed at the cytoplasmic DNA-based STING pathway. In light of this pharmacologic activation of IRF3/IFN via STING may represent an efficacious therapeutic strategy. Disclosed herein is the identification and characterization of a small molecule capable of stimulating IRF3 phosphorylation and IFN production in human cells that prevents replication of Alphaviruses. Reverse genetic studies using CRISPR/Cas9-mediated gene editing are also disclosed that show that this molecule requires STING for its innate gene induction and antiviral activity and thus it represents the first synthetic compound definitively capable of activating this pathway in human cells. Moreover, in vivo stimulation of the STING pathway was also shown to prevent replication of CHIKV demonstrating the potential therapeutic application of pharmacologically targeting activation of this protein.

### SUMMARY

STING is a pattern recognition receptor of cyclic dinucleotides as well as an innate immune adaptor protein that enables signaling from cytoplasmic receptors to the transcription factor interferon regulatory factor 3. Initiation of these pathways leads to the expression of type I interferons and proteins associated with antiviral and antitumor immunity. Small molecules capable of triggering STING-dependent cellular processes are effective at blocking virus replication, enhancing vaccine efficacy, and facilitating an immune response to cancer cells.

Disclosed herein is the first synthetic small molecule capable of activating STING-mediated signaling in human cells. Also disclosed is that exposure of cells to the compound renders them refractory to replication by interferon-sensitive emerging Alphaviruses. In addition, in vivo stimulation of STING dependent activity also blocks viremia of Chikungunya virus. Ultimately this work may lead to the utilization of STING as a target for multiple immune-mediated therapies.

Disclosed herein is the identification and characterization of a small molecule capable of stimulating IRF3 phosphorylation and IFN production in human cells that prevents replication of Alphaviruses. Through reverse genetic studies using CRISPR/Cas9-mediated gene editing it was shown that this molecule requires STING for its innate gene induction and antiviral activity and thus it represents the first synthetic compound definitively capable of activating this pathway in human cells. Moreover, in vivo stimulation of the STING pathway was also shown to prevent replication of CHIKV demonstrating the potential therapeutic application of pharmacologically targeting activation of this protein.

Disclosed are methods of identifying a test compound that is likely to act as an agonist of one or more proteins in the STING pathway. The methods involve contacting a first transfected human cell with the test compound. The first transfected human cell includes an expression vector. The expression vector includes (i) a polynucleotide encoding a human telomerase reverse transcriptase with expression driven by a constitutively active promoter and (ii) a polynucleotide encoding a bioluminescent or fluorescent protein with expression driven by a promoter that promotes expression in the presence of interferon regulatory factor 3. The first transfected human cell expresses STING. The method further involves contacting a second transfected human cell with the test compound. The second transfected human cell includes the same expression vector as the first transfected human cell, but the second transfected human cell does not express STING. Expression of the bioluminescent or fluorescent protein in the presence of the test compound in the first transfected human cell line that is greater than the expression of the bioluminescent or fluorescent protein in the presence of the test compound in the second transfected human cell line is an indication that the test compound is likely to act as an agonist of one or more proteins in the STING pathway.

In embodiments, the methods can further involve the second promoter also promoting expression in the presence of type I interferon. The second transfected human cell line can lack expression of STING due to the excising of the STING gene using CRISPR/Cas9. The bioluminescent or fluorescent protein can be a luciferase. The methods can further involve assessing whether or not the test compound is not likely to induce NF-κB. The methods can further involve use of a test compound that acts as a positive control including a test compound with the formula:

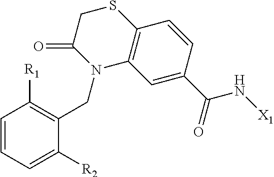

where X₂ is aryl or aryl substituted alkyl, and where R₁ and R₂ are independently H or halo.

Such positive control compounds include a compound with the formula:

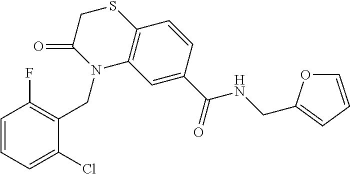 [SMILES: O=C(NCc1ccco1)c1ccc2c(c1)N(Cc1c(F)cccc1Cl)C(=O)CS2]

Disclosed are compounds with the formula:

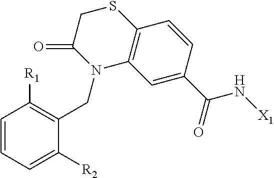

where X₂ is aryl or aryl substituted alkyl, and where R₁ and R₂ are independently H or halo. In embodiments, the disclosed compounds include compounds with the formula:

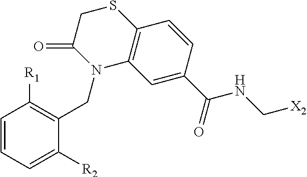

where X₂ is aryl. In still further embodiments, X₂ can be phenyl or furanyl, and can include compounds with the formula:

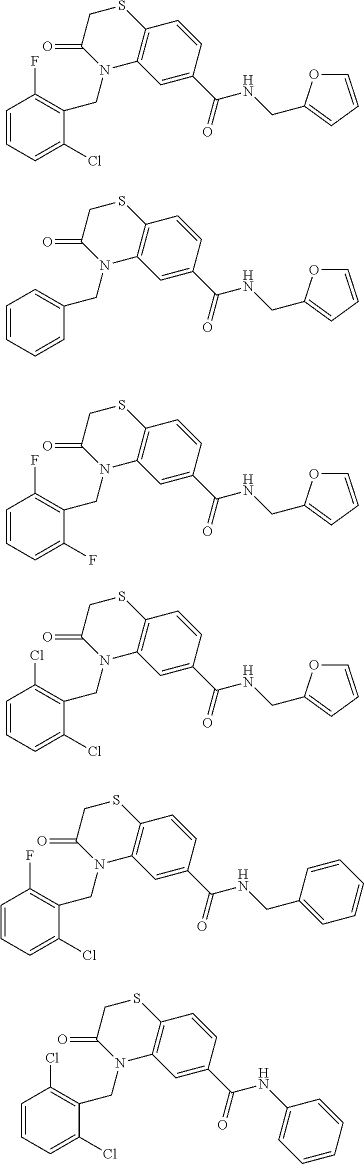

Also disclosed are pharmaceutical compositions comprising the above compounds and a pharmaceutically acceptable carrier.

Also disclosed are methods of inhibiting alphavirus replication in a subject. Such methods involve administering the above pharmaceutical composition to the subject, thereby inhibiting the alphavirus replication. In still further embodiments, the pharmaceutical composition comprises the compound designated G10 herein. In still further embodiments, the alphavirus is chikungunya virus.

### BRIEF DESCRIPTION OF THE SEVERAL VIEWS OF THE DRAWINGS

FIG. 1A is a graph showing values representing the average±SD of duplicate assays. Black bar and arrow indicate LUC signal generated by G10.

FIG. 1B is a chemical structure of 4-(2-chloro-6-fluorobenzyl)-N-(furan-2-ylmethyl)-3-oxo-3,4-dihydro-2H1290 benzo[b][1,4]thiazine-6-carboxamide (G10).

FIG. 2A is a graph showing dose-dependent expression of IRF3/IFN-dependent luciferase (LUC) in telomerized human fibroblasts (THF). Values displayed are average fold changes±SD of quadruplicate measurements of luminescence following 7 h exposure to indicated concentration of G10 relative to cells exposed only to 1% DMSO (all samples normalized to 1% DMSO).

FIG. 2B is a graph showing mRNA transcription of genes dependent on IRF3/IFN following 7 h exposure to 100 μM G10 or UV-CMV. Indicated values represent average±SEM mRNA fold change relative to cells exposed to 1% DMSO from duplicate experiments.

FIG. 2C is a graph showing Induction of NF-κB-dependent LUC signal in THF reporter cells following 7 h exposure to 1 μg/mL LPS, 160 HA units/mL SeV, 1 ng/mL TNFα or indicated concentration of G10. Values displayed are as described in (FIG. 2A).

FIG. 2D is a graph showing mRNA transcription of NF-κB-dependent genes following 7 h exposure to 100 μM G10 or 160 HA units/mL SeV. Indicated values are mRNA fold change relative to cells exposed to 1% DMSO and are representative of duplicate experiments.

FIG. 3 is a set of four plots showing average titers±SD of VACV, WNV, CHIKV, and VEEV grown on THF cells in the presence of 1308 indicated G10 concentration (DMSO concentration normalized to 1%). Infections were performed in triplicate and virus harvested at 48 h post infection (CHIKV, WNV, CHIKV) or 24 h post infection (VACV, VEEV).

FIG. 4A is an immunoblot showing IRF3, STAT1, and GAPDH in THF-ISRE stably transduced with Cas9 and CRISPR gRNA directed against either STAT1 (THF-ISRE-ΔSTAT1) or IRF3 (THF-ISRE-ΔIRF3) as indicated.

FIG. 4B is a graph showing the Induction of IRF3/IFN-dependent LUC in THF lacking STAT1 following 7 h exposure to 100 μM G10, UV-inactivated CMV, or 1000 U/mL IFNβ. Values displayed are average fold changes±SD of quadruplicate measurements relative to cells exposed only to 1% DMSO.

FIG. 4C is a graph showing the induction of IRF3/IFN dependent LUC in THF lacking IRF3 following 7 h exposure to 100 μM G10, UV inactivated CMV, or 1000 U/mL IFNβ. Values displayed are as in FIG. 4B.

FIG. 4D is an immunoblot of lysates from THF-ISRE following 6 h exposure to DMSO, UV-CMV, SeV or 100 uM G10 as indicated showing phosphorylation status of IRF3 S386, total IRF3, and GAPDH.

FIG. 4E is a set of three graphs showing average media titers+SD of CHIKV, VEEV, and SINV at 24 (VEEV) or 48 hpi (CHIKV, SINV) obtained from THF-ISRE-ΔIRF3 cells treated with 1% DMSO, 100 μM G10, or 1000 U/mL IFNβ as indicated. Infections were performed in triplicate.

FIG. 5A is an immunoblot of lysates from THF-ISRE-ΔIPS1 following 6 h exposure to DMSO, UV-CMV, SeV or 100 uM G10 as indicated showing phosphorylation status of IRF3 S386, total IRF3, IPS1, STING, and GAPDH.

FIG. 5B is a set of three graphs showing average media titers+SD of CHIKV, VEEV, and SINV at 24 h (VEEV) or 48 h (CHIKV, SINV) post infection obtained from THF-ISRE-ΔIPS1 cells treated with 1% DMSO, 100 μM G10, or 1000 U/mL IFNβ as indicated. Infections were performed in triplicate.

FIG. 6A is an immunoblot of lysates from THF-ΔSTING following 7 h exposure to 1% DMSO, 0.1 μg/mL poly(I:C), SeV, UV-CMV, 1 μg/mL 2′3′-cGAMP, or 100 uM G10 as indicated showing phosphorylation status of IRF3 S386, total IRF3, IPS1, STING, and GAPDH.

FIG. 6B is a graph showing expression of IRF3/IFN dependent LUC in THF-ISRE-ΔIPS1 and THF-ISRE-ΔSTING following 7 hours exposure to 1% DMSO, indicated concentrations of G10, SeV, UV-CMV, or 1 μg/mL LPS. Values are presented as average fold change in quadruplicate measurements±SD relative to cells treated with 1% DMSO.

FIG. 6C is a set of three graphs of the average fold changes±SD from duplicate experiments of ISG54, ISG15, and Viperin mRNA relative to cells treated with 1% DMSO in THF-ISRE-ΔIPS1 (black bars) or THF-ISRE-ΔSTING (gray bars) following exposure to UV-CMV, SeV, or 100 μM G10.

FIG. 6D is a set of two graphs showing media titers of CHIKV and VEEV at 24 h (VEEV) or 48 h (CHIKV) obtained from THF-ISRE-ΔSTING cells treated with 1% DMSO, 100 μM G10, or 1000 U/mL IFNβ as indicated. Infections were performed in triplicate.

FIG. 7A is a set of two graphs showing Induction of IFNβ and IFNλ1 transcripts in THF following 18 hours of exposure to SeV, UV-CMV, or 100 μM G10 or IFNβ as indicated. Values presented are average fold changes relative to cells treated with 1% DMSO±SD based on duplicate treatments.

FIG. 7B is graphs showing induction of Mx2 and OAS transcripts in THF following 18 h exposure to SeV, UV-CMV, or 100 μM G10 or IFNβ as indicated. Values presented are average fold changes relative to cells treated with 1% DMSO±SD based on duplicate treatments.

FIG. 7C is a set of three graphs showing secretion of type I IFN from THF-ISRE, THF-ISRE-ΔIPS1, or THF-ISRE-ΔSTING following 18 hours of exposure to 1% DMSO, SeV, UV-CMV, or 100 μM G10. Average LUC values±SD were obtained from THF-ISRE-ΔIRF3 exposed (in triplicate) to media harvested from indicated cells and exposed to indicated stimulus (in triplicate).

FIG. 8A is a graph of the average media titers±SD of CHIKV, VEEV, and SINV at 24 hours (VEEV) or 48 hours (CHIKV, SINV) obtained from THF-ISRE-ΔSTAT1 cells treated with 1% DMSO, 100 μM G10, or 1000 U/mL IFNβ as indicated. Infections were performed in triplicate.

FIG. 8B is an immunoblot of the synthesis of Mx2 and ISG56 proteins in THF-ISRE and THF-ISRE-ΔSTAT1 following 24 h exposure to SeV, UV-CMV, 1000 U/mL IFNβ or 100 μM G10 as indicated.

FIG. 9A is an immunoblot of lysates from THF-ISRE cells following exposure to G10 (100 μM) or transfected 2′3′-cGAMP (42.3 μM) for indicated time showing phosphorylation status of IRF3 S386, total IRF3, and GAPDH.

FIG. 9B is a set of seven plots showing mRNA synthesis of indicated genes in THF following 8 h exposure to indicated concentration of G10 (blue) or 2′3′-cGAMP (red). Indicated values represent average mRNA fold change±SD from duplicate experiments relative to cells exposed to 1% DMSO.

FIG. 10A is a set of 8 graphs showing mRNA synthesis of indicated genes in human peripheral blood mononuclear cells (PBMC) following 8 h exposure to indicated concentration of G10 ppp-dsRNA (12.5 μg/mL)

FIG. 10B is a set of 8 graphs showing mRNA synthesis of indicated genes in human PBMC following 8 hours exposure to the indicated concentration of 2′3′-cGAMP (28 μM). Indicated values represent average mRNA fold change±SD from duplicate experiments relative to cells exposed to 1% DMSO.

FIG. 11A is an immunoblot of lysates from murine RAW264.7 cells following exposure to 100 μM DMXAA for indicated time showing phosphorylation status of IRF3 S386, total IRF3, and GAPDH.

FIG. 11B is a set of four plots of mRNA synthesis of indicated genes in RAW264.7 cells following 8 h exposure to indicated concentration of DMXAA. Values 1386 represent average mRNA fold change±SD from duplicate experiments relative to cells exposed to 1% DMSO.

FIG. 11C is a plot of levels of serum-associated CHIKV at 72 h post infection. Five mice per group were treated with DMSO alone or DMXAA at 3 h pre- or 6 h or 24 h post inoculation as indicated.

FIG. 12 is a graph of ATP-dependent luminescence of THF following 24 hr or 48 hr exposure to indicated concentrations of G10 or 3% DMSO (0). Values displayed are raw luminescence values averaged from quadruplicate measurements±SD following 24 h or 48 h exposure to indicated concentration of G10.

FIG. 13 is a graph of average media titers+SD of SINV at 24 h or 48 h post infection on wild type THF-ISRE cells and at 24 h post infection of cells lacking IPS-1 as indicated Infections were performed in triplicate.

FIG. 14A is a graph of THP1-ISG-Lucia cells differentiated for 24 h with 100 nM PMA were treated overnight with 1% DMSO, G10 at indicated concentration, UV-CMV, or SeV. Expression of Lucia luciferase was quantitated by measuring luminescence from quadruplicate treatments. Data illustrated are average Lucia fold changes±SD calculated relative to DMSO-treated cells.

FIG. 14B is a graph of mRNA synthesis of indicated genes in differentiated THP-1 cells following 8 h exposure to SeV, UV-CMV, or 100 μM G10. Indicated values illustrate mRNA fold change and are representative of duplicate experiments relative to untreated cells.

FIG. 15 is a set of graphs of mRNA synthesis of indicated genes in human umbilical microvascular endothelial cells following 8 h exposure to UV-CMV or 100 μM G10. Indicated values represent average mRNA fold change±SD from duplicate experiments relative to cells exposed to 1% DMSO.

FIG. 16 is a graph of luminescence detected in RAW264.7 cells stably transduced with IFN1430 dependent LUC (RAW264.7-ISRE) following overnight exposure to 1% DMSO or indicated concentration of DMXAA or G10.

FIG. 17 is a graph showing the ability of G10 analogs to induce luciferase signal in THF-ISRE. Data illustrated are average LUC fold changes±SD calculated relative to DMSO-treated cells for quadruplicate treatments.

### SEQUENCE LISTING

SEQ ID NO: 1 is a protein sequence of human STING.

SEQ ID NO: 2 is a protein sequence of human telomerase reverse transcriptase.

SEQ ID NO: 3 is a protein sequence of firefly luciferase protein.

SEQ ID NO: 4 is a protein sequence of human regulatory factor 3 protein.

SEQ ID NO: 5 is a nucleic acid sequence of human ISG154.

SEQ ID NO: 6 is a nucleic acid sequence of human ISG156.

SEQ ID NO: 7 is a nucleic acid sequence of human ISG15.

SEQ ID NO: 8 is a nucleic acid sequence of human VIPERIN.

SEQ ID NO: 9 is a protein sequence of human NF-κB.

SEQ ID NO: 10 is a nucleic acid sequence of human IL-8.

SEQ ID NO: 11 is a nucleic acid sequence of human IL-1β.

SEQ ID NO: 12 is a nucleic acid sequence of human MIP-1α.

SEQ ID NOs: 13-32 are oligonucleotide primers.

### DETAILED DESCRIPTION

### Terms

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below.

As will be understood by one of ordinary skill in the art, each embodiment disclosed herein can comprise, consist essentially of, or consist of its particular stated element, step, ingredient or component. Thus, the terms “include” or “including” should be interpreted to recite: “comprise, consist essentially of, or consist of.” The transition term “comprise” or “comprises” means includes, but is not limited to, and allows for the inclusion of unspecified elements, steps, ingredients, or components, even in major amounts. The transitional phrase “consisting essentially of” limits the scope of the embodiment to the specified elements, steps, ingredients or components and to those that do not materially affect the embodiment. The transitional phrase “consisting of” excludes any element, step, ingredient or component not specified.

Agonist: An agonist is an agent, such as a small molecule or protein that binds to a protein and causes, enhances, or augments (to a statistically significant degree) a particular biological effect of the protein. Agonists can be naturally occurring or artificially synthesized compounds. For example, an agonist of a protein in the STING pathway is a compound that augments the natural activity of a protein in the STING pathway (either upstream or downstream).

Alkyl: a branched or unbranched saturated hydrocarbon group, such as methyl, ethyl, n-propyl, isopropyl, n-butyl, isobutyl, t-butyl, pentyl, hexyl, heptyl, octyl, nonyl, decyl, tetradecyl, hexadecyl, eicosyl, tetracosyl and the like. A lower alkyl group is a saturated branched or unbranched hydrocarbon having from 1 to 6 carbon atoms (C1-6 alkyl). The term alkyl also includes cycloalkyls. Alkyl also includes substituted alkyls which are alkyl groups wherein one or more hydrogen atoms are replaced with a substituent such as alkyl, alkynyl, alkenyl, aryl, halide, nitro, amino, ester, ether, ketone, aldehyde, hydroxyl, carboxyl, cyano, amido, haloalkyl, haloalkoxy, or alkoxy. The term alkyl also includes heteroalkyls. A heteroalkyl contains at least one heteroatom such as nitrogen, oxygen, sulfur, or phosphorus replacing one or more of the carbons. Substituted heteroalkyls are also encompassed by the term alkyl.

Aryl: any carbon-based aromatic group including benzene, naphthalene, and phenyl. The term aryl also includes substituted aryls in which one or more of the hydrogens is substituted with one or more groups including alkyl, alkynyl, alkenyl, aryl, halide, nitro, amino, ester, ether, ketone, aldehyde, hydroxy, carboxylic acid, cyano, amido, haloalkyl, haloalkoxy, or alkoxy. The term aryl also includes heteroaryls in which one or more of the carbons is replaced by a heteroatom. Examples of heteroatoms include nitrogen, oxygen, sulfur, and phosphorous. Substituted heteroaryls are also encompassed by the term aryl.

Bioluminescent proteins or photoproteins: proteins derived from bioluminescent organisms that emit light by the conversion of chemical bond energy to light energy. Examples of such proteins include luciferase from any source including Aqueora victoria, or Phytonis pyralis.

Contacting: Placement under conditions in which direct physical association occurs, including contacting of a solid with a solid, a liquid with a liquid, a liquid with a solid, or either a liquid or a solid with a cell or tissue, whether in vitro or in vivo. Contacting can occur in vitro with isolated cells or tissue or in vivo by administering to a subject.

Control: A reference standard. A control can be a test compound that is known to be an agonist of a protein in the STING pathway (positive control), such as G10. A control can also be a test compound known not to act as an agonist of a protein in the STING pathway, such as the vehicle in which the test compound is provided, otherwise lacking the test compound (negative control).

Derivative: a compound or portion of a compound that is derived from or is theoretically derivable from a parent compound. Within the current disclosure, a derivative exhibits a substantially similar biological effect in the methods disclosed and claimed herein.

Effective Amount: An amount of an agent that is sufficient to generate a desired response such as reducing or eliminating a sign or symptom of a condition or a disease. An effective amount also encompasses an effective amount of a first agent and an effective amount of a second agent administered in combination with the first agent. In some examples, the effective amount of the two combined agents is less than that of either agent when administered alone.

Fluorescent protein: A protein characterized by a barrel structure that allows the protein to absorb light and emit it at a particular wavelength. Fluorescent proteins include green fluorescent protein (GFP) modified GFPs and GFP derivatives and other fluorescent proteins, such as EGFP, EBFP, YFP, BFP, CFP, ECFP, and circularly permutated fluorescent proteins such as cpVenus.

Heterocycle: A chemical group that includes both heteroaryls and heterocycloalkyls. Heterocycles may be monocyclic or polycyclic rings. Exemplary heterocycles include azepinyl, aziridinyl, azetyl, azetidinyl, diazepinyl, dithiadiazinyl, dioxazepinyl, dioxolanyl, dithiazolyl, furanyl, isooxazolyl, isothiazolyl, imidazolyl, morpholinyl, oxetanyl, oxadiazolyl, oxiranyl, oxazinyl, oxazolyl, piperazinyl, pyrazinyl, pyridazinyl, pyrimidinyl, piperidyl, piperidino, pyridyl, pyranyl, pyrazolyl, pyrrolyl, pyrrolidinyl, thiatriazolyl, tetrazolyl, thiadiazolyl, triazolyl, thiazolyl, thienyl, tetrazinyl, thiadiazinyl, triazinyl, thiazinyl, thiopyranyl, furoisoxazolyl, imidazothiazolyl, thienoisothiazolyl, thienothiazolyl, imidazopyrazolyl, cyclopentapyrazolyl, pyrrolopyrrolyl, thienothienyl, thiadiazolopyrimidinyl, thiazolothiazinyl, thiazolopyrimidinyl, thiazolopyridinyl, oxazolopyrimidinyl, oxazolopyridyl, benzoxazolyl, benzisothiazolyl, benzothiazolyl, imidazopyrazinyl, purinyl, pyrazolopyrimidinyl, imidazopyridinyl, benzimidazolyl, indazolyl, benzoxathiolyl, benzodioxolyl, benzodithiolyl, indolizinyl, indolinyl, isoindolinyl, furopyrimidinyl, furopyridyl, benzofuranyl, isobenzofuranyl, thienopyrimidinyl, thienopyridyl, benzothienyl, cyclopentaoxazinyl, cyclopentafuranyl, benzoxazinyl, benzothiazinyl, quinazolinyl, naphthyridinyl, quinolinyl, isoquinolinyl, benzopyranyl, pyridopyridazinyl and pyridopyrimidinyl groups. The term also includes substituted heterocycles, including substituted forms of all the species above.

Label: A label may be any substance capable of aiding a machine, detector, sensor, device, column, or enhanced or unenhanced human eye in differentiating a labeled composition from an unlabeled composition. Labels may be used for any of a number of purposes and one skilled in the art will understand how to match the proper label with the proper purpose. Examples of uses of labels include purification of biomolecules, identification of biomolecules, detection of the presence of biomolecules, detection of protein folding, and localization of biomolecules within a cell, tissue, or organism. Examples of labels include: radioactive isotopes or chelates thereof; dyes (fluorescent or nonfluorescent), stains, enzymes, nonradioactive metals, magnets, protein tags, any antibody epitope, any specific example of any of these; any combination between any of these, or any label now known or yet to be disclosed. A label may be covalently attached to a biomolecule or bound through hydrogen bonding, Van Der Waals or other forces.

One particular example of a label is a protein tag. A protein tag includes a sequence of one or more amino acids that may be used as a label as discussed above, particularly for use in protein purification. In some examples, the protein tag is covalently bound to the polypeptide. It may be covalently bound to the N-terminal amino acid of a polypeptide, the C-terminal amino acid of a polypeptide or any other amino acid of the polypeptide. Often, the protein tag is encoded by a polynucleotide sequence that is immediately 5′ of a nucleic acid sequence coding for the polypeptide such that the protein tag is in the same reading frame as the nucleic acid sequence encoding the polypeptide. Protein tags may be used for all of the same purposes as labels listed above and are well known in the art. Examples of protein tags include chitin binding protein (CBP), maltose binding protein (MBP), glutathione-S-transferase (GST), poly-histidine (His), thioredoxin (TRX), FLAG®, V5, c-Myc, HA-tag, and so forth.

A His-tag facilitates purification and binding to on metal matrices, including nickel matrices, including nickel matrices bound to solid substrates such as agarose plates or beads, glass plates or beads, or polystyrene or other plastic plates or beads. Other protein tags include BCCP, calmodulin, Nus, Thioredoxin, Streptavidin, SBP, and Ty, or any other combination of one or more amino acids that can work as a label described above.

Another particular example of a label is biotin. Biotin is a natural compound that tightly binds proteins such as avidin or streptavidin. A compound labeled with biotin is said to be ‘biotinylated’. Biotinylated compounds can be detected with avidin or streptavidin when that avidin or streptavidin is conjugated another label such as a fluorescent, enzymatic, radioactive or other label.

Operably Linked: A first nucleic acid sequence is operably linked with a second nucleic acid sequence when the first nucleic acid sequence is placed in such a way that it has an effect upon the second nucleic acid sequence. For instance, a promoter is operably linked to a coding sequence if the promoter affects the transcription or expression of the coding sequence. Operably linked DNA sequences may be contiguous, or they may operate at a distance.

Polynucleotide: a polymer of ribonucleic acid (RNA) or deoxyribonucleic acid (DNA). The term can be used interchangeably with the term ‘nucleic acid.’ A polynucleotide is made up of four bases; adenine, cytosine, guanine, and thymine/uracil (uracil is used in RNA). A coding sequence from a nucleic acid is indicative of the sequence of the protein encoded by the nucleic acid.

Polypeptide: Any chain of amino acids, regardless of length or posttranslational modification (such as glycosylation, methylation, ubiquitination, phosphorylation, or the like). Herein as well as in the art, the term ‘polypeptide’ is used interchangeably with peptide or protein, and is used to refer to a polymer of amino acid residues. The term ‘residue’ can be used to refer to an amino acid or amino acid mimetic incorporated in a polypeptide by an amide bond or amide bond mimetic. Polypeptide sequences are generally written with the N-terminal amino acid on the left and the C-terminal amino acid to the right of the sequence.

Promoter: A promoter may be any of a number of nucleic acid control sequences that directs transcription of a nucleic acid. Typically, a eukaryotic promoter includes necessary nucleic acid sequences near the start site of transcription, such as, in the case of a polymerase II type promoter, a TATA element or any other specific DNA sequence that is recognized by one or more transcription factors. Expression by a promoter may be further modulated by enhancer or repressor elements. Numerous examples of promoters are available and well known to those of skill in the art. A nucleic acid including a promoter operably linked to a nucleic acid sequence that codes for a particular polypeptide can be termed an expression vector.

Recombinant: A recombinant nucleic acid or polypeptide has a sequence that is not naturally occurring or has a sequence that is made by an artificial combination of two or more naturally occurring sequences. This artificial combination is often accomplished by chemical synthesis or, more commonly, by the artificial manipulation of isolated segments of nucleic acids, e.g., by genetic engineering techniques. A recombinant polypeptide can refer to a polypeptide that has been made using recombinant nucleic acids, including recombinant nucleic acids transferred to a host organism that is not the natural source of the polypeptide.

Subject: A living multicellular vertebrate organism, a category that includes, for example, mammals and birds. A “mammal” includes both human and non-human mammals, such as mice. In some examples, a subject is a patient, such as a patient diagnosed with cancer. In other examples, a subject is a patient yet to be diagnosed with cancer.

Test Compound: A test compound can be any compound that is suspected of or might be an agonist of one or more proteins in the STING pathway. Examples of test compounds include small molecules, proteins, peptides, or other potential therapeutic compounds. A test compound can also be a compound known to be an agonist of the STING pathway that is used as a positive control. A test compound can also be a compound known not to affect the activity of the STING pathway that is used as a negative control.

Treatment: any therapeutic intervention that ameliorates a sign or symptom of a disease or pathological condition. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of metastases, an improvement in the overall health or well-being of the subject, or by other clinical or physiological parameters associated with a particular disease. A “prophylactic” treatment is a treatment administered to a subject who does not exhibit signs of a disease or exhibits only early signs for the purpose of decreasing the risk of developing pathology. A “therapeutic” treatment is a treatment administered after the development of significant signs or symptoms of the disease.

### Compounds:

Disclosed are compounds of the formula:

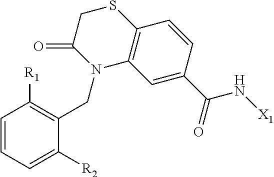

where R₁ and R₂ are independently H or halo and where X₁ is aryl or aryl substituted alkyl.

Also disclosed are compounds of the formula:

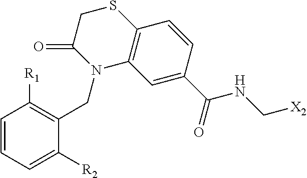

where X₂ is aryl. In some embodiments, X₂ is benzyl, substituted benzyl, 5 membered heterocycle or substituted 5 membered heterocyle. In still further embodiments, X₂ is furanyl. Particular examples of the disclosed compounds include:

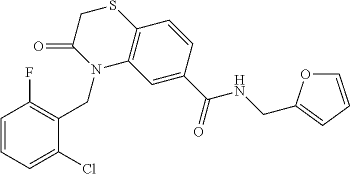 [SMILES: O=C(NCc1ccco1)c1ccc2c(c1)N(Cc1c(F)cccc1Cl)C(=O)CS2]

which is referred to herein as G10;

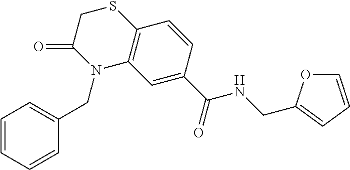 [SMILES: O=C(NCc1ccco1)c1ccc2c(c1)N(Cc1ccccc1)C(=O)CS2]

which is referred to herein as G10-01;

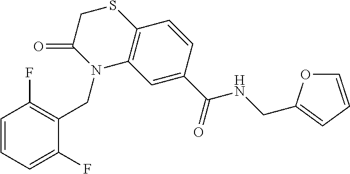 [SMILES: O=C(NCc1ccco1)c1ccc2c(c1)N(Cc1c(F)cccc1F)C(=O)CS2]

which is referred to herein as G10-02;

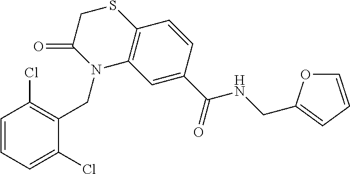 [SMILES: O=C(NCc1ccco1)c1ccc2c(c1)N(Cc1c(Cl)cccc1Cl)C(=O)CS2]

which is referred to as G10-03

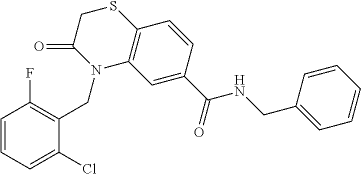 [SMILES: O=C(NCc1ccccc1)c1ccc2c(c1)N(Cc1c(F)cccc1Cl)C(=O)CS2]

which is referred to as G10-04;

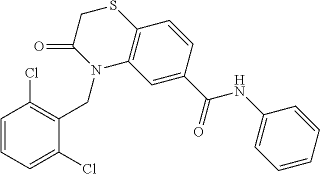 [SMILES: O=C(Nc1ccccc1)c1ccc2c(c1)N(Cc1c(Cl)cccc1Cl)C(=O)CS2]

which is referred to as G10-05.

### Pharmaceutical Compositions

The compounds disclosed herein may be included in pharmaceutical compositions (including therapeutic and prophylactic formulations), typically combined together with one or more pharmaceutically acceptable carriers (known equivalently as vehicles) and, optionally, other therapeutic ingredients.

Such pharmaceutical compositions can formulated for administration to subjects by a variety of mucosal administration modes, including by oral, rectal, intranasal, intrapulmonary, intravitrial, or transdermal delivery, or by topical delivery to other surfaces including the eye. Optionally, the compositions can be administered by non-mucosal routes, including by intramuscular, subcutaneous, intravenous, intra-arterial, intra-articular, intraperitoneal, intrathecal, intracerebroventricular, or parenteral routes. In other examples, the compound can be administered ex vivo by direct exposure to cells, tissues or organs originating from a subject.

To formulate the pharmaceutical compositions, the compound can be combined with various pharmaceutically acceptable additives. Desired additives include, but are not limited to, pH control agents, such as arginine, sodium hydroxide, glycine, hydrochloric acid, citric acid, and the like. In addition, local anesthetics (for example, benzyl alcohol), isotonizing agents (for example, sodium chloride, mannitol, sorbitol), adsorption inhibitors (for example, Tween®-80), solubility enhancing agents (for example, cyclodextrins and derivatives thereof), stabilizers (for example, serum albumin), and reducing agents (for example, glutathione) can be included.

When the composition is a liquid, the tonicity of the formulation, as measured with reference to the tonicity of 0.9% (w/v) physiological saline solution taken as unity, is typically adjusted to a value at which no substantial, irreversible tissue damage will be induced at the site of administration. Generally, the tonicity of the solution is adjusted to a value of about 0.3 to about 3.0, such as about 0.5 to about 2.0, or about 0.8 to about 1.7. The compound can be dispersed in any pharmaceutically acceptable carrier, which can include a hydrophilic compound having a capacity to disperse the compound, and any desired additives. The carrier can be selected from a wide range of suitable compounds, including but not limited to, copolymers of polycarboxylic acids or salts thereof, carboxylic anhydrides (for example, maleic anhydride) with other monomers (for example, methyl (meth)acrylate, acrylic acid and the like), hydrophilic vinyl polymers, such as polyvinyl acetate, polyvinyl alcohol, polyvinylpyrrolidone, cellulose derivatives, such as hydroxymethylcellulose, hydroxypropylcellulose and the like, and natural polymers, such as chitosan, collagen, sodium alginate, gelatin, hyaluronic acid, and nontoxic metal salts thereof. Often, a biodegradable polymer is selected as a carrier, for example, polylactic acid, poly(lactic acid-glycolic acid) copolymer, polyhydroxybutyric acid, poly(hydroxybutyric acidglycolic acid) copolymer and mixtures thereof.

Alternatively or additionally, synthetic fatty acid esters such as polyglycerin fatty acid esters, sucrose fatty acid esters and the like can be employed as carriers. Hydrophilic polymers and other vehicles can be used alone or in combination, and enhanced structural integrity can be imparted to the vehicle by partial crystallization, ionic bonding, cross-linking and the like. The carrier can be provided in a variety of forms, including fluid or viscous solutions, gels, pastes, powders, microspheres, and films for direct application to a mucosal surface.

The compound can be combined with the carrier according to a variety of methods, and release of the compound can be by diffusion, disintegration of the vehicle, or associated formation of water channels. In some circumstances, the compound is dispersed in microcapsules (microspheres) or nanoparticles prepared from a suitable polymer, for example, 5-isobutyl 2-cyanoacrylate (see, for example, Michael et al., J. Pharmacy Pharmacol. 43, 1-5, (1991)), and dispersed in a biocompatible dispersing medium, which yields sustained delivery and biological activity over a protracted time.

Pharmaceutical compositions for administering the compound can also be formulated as a solution, microemulsion, or other ordered structure suitable for high concentration of active ingredients. The vehicle can be a solvent or dispersion medium containing, for example, water, ethanol, polyol (for example, glycerol, propylene glycol, liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants. In many cases, it will be desirable to include isotonic agents, for example, sugars, polyalcohols, such as mannitol and sorbitol, or sodium chloride in the composition. Prolonged absorption of the compound can be brought about by including in the composition an agent which delays absorption, for example, monostearate salts and gelatin.

In certain embodiments, the compound can be administered in a time release formulation, for example in a composition which includes a slow release polymer. These compositions can be prepared with vehicles that will protect against rapid release, for example a controlled release vehicle such as a polymer, microencapsulated delivery system or bioadhesive gel. Prolonged delivery in various compositions of the disclosure can be brought about by including in the composition agents that delay absorption, for example, aluminum monostearate hydrogels and gelatin. When controlled release formulations are desired, controlled release binders suitable for use in accordance with the disclosure include any biocompatible controlled release material which is inert to the active agent and which is capable of incorporating the compound and/or other biologically active agent. Numerous such materials are known in the art. Useful controlled-release binders are materials that are metabolized slowly under physiological conditions following their delivery (for example, at a mucosal surface, or in the presence of bodily fluids). Appropriate binders include, but are not limited to, biocompatible polymers and copolymers well known in the art for use in sustained release formulations. Such biocompatible compounds are non-toxic and inert to surrounding tissues, and do not trigger significant adverse side effects, such as nasal irritation, immune response, inflammation, or the like. They are metabolized into metabolic products that are also biocompatible and easily eliminated from the body.

Exemplary polymeric materials for use in the present disclosure include, but are not limited to, polymeric matrices derived from copolymeric and homopolymeric polyesters having hydrolyzable ester linkages. A number of these are known in the art to be biodegradable and to lead to degradation products having no or low toxicity. Exemplary polymers include polyglycolic acids and polylactic acids, poly(DL-lactic acidco-glycolic acid), poly(D-lactic acid-co-glycolic acid), and poly(L-lactic acid-coglycolic acid). Other useful biodegradable or bioerodable polymers include, but are not limited to, such polymers as poly(epsilon-caprolactone), poly(epsilon-aprolactone-CO-lactic acid), poly(epsilon.-aprolactone-CO-glycolic acid), poly(betahydroxy butyric acid), poly(alkyl-2-cyanoacrilate), hydrogels, such as poly(hydroxyethyl methacrylate), polyamides, poly(amino acids) (for example, L-leucine, glutamic acid, L-aspartic acid and the like), poly(ester urea), poly(2-hydroxyethyl DL-aspartamide), polyacetal polymers, polyorthoesters, polycarbonate, polymaleamides, polysaccharides, and copolymers thereof. Many methods for preparing such formulations are well known to those skilled in the art (see, for example, Sustained and Controlled Release Drug Delivery Systems, J. R. Robinson, ed., Marcel Dekker, Inc., New York, 1978). Other useful formulations include controlled-release microcapsules (U.S. Pat. Nos. 4,652,441 and 4,917,893), lactic acid-glycolic acid copolymers useful in making microcapsules and other formulations (U.S. Pat. Nos. 4,677,191 and 4,728,721) and sustained-release compositions for water-soluble peptides (U.S. Pat. No. 4,675,189).

The pharmaceutical compositions of the disclosure typically are sterile and stable under conditions of manufacture, storage and use. Sterile solutions can be prepared by incorporating the compound in the required amount in an appropriate solvent with one or a combination of ingredients enumerated herein, as required, followed by filtered sterilization. Generally, dispersions are prepared by incorporating the compound and/or other biologically active agent into a sterile vehicle that contains a basic dispersion medium and the required other ingredients from those enumerated herein. In the case of sterile powders, methods of preparation include vacuum drying and freeze-drying which yields a powder of the compound plus any additional desired ingredient from a previously sterile-filtered solution thereof. The prevention of the action of microorganisms can be accomplished by various antibacterial and antifungal agents, for example, parabens, chlorobutanol, phenol, sorbic acid, thimerosal, and the like.

### Treatment

Disclosed are methods of treating a subject with an alphavirus infection using combinations of compositions described herein. The compounds can be administered by any appropriate route including orally or parenterally including buccally, sublingually, sublabially, by inhalation, intra-arterially, intravenously, intraventricularly, intramuscularly, subcutaneously, intraspinally, intraorbitally, intracranially or intrathecally.

The administration of a pharmaceutical composition comprising the disclosed compounds can be for prophylactic or therapeutic purposes. For prophylactic and therapeutic purposes, the treatments can be administered to the subject in a single bolus delivery, via continuous delivery (for example, continuous transdermal, mucosal or intravenous delivery) over an extended time period, or in a repeated administration protocol (for example, by an hourly, daily or weekly, repeated administration protocol). The therapeutically effective dosage of the treatments for viral infection can be provided as repeated doses within a prolonged prophylaxis or treatment regimen that will yield clinically significant results to alleviate one or more symptoms or detectable conditions associated with a disease or condition.

An effective amount or concentration of the disclosed combinations of compounds can be any amount of the two compounds administered by themselves alone or in combination with additional therapeutic agents, is sufficient to achieve a desired effect in a subject. The effective amount of the agent will be dependent on several factors, including, but not limited to, the subject being treated and the manner of administration of the compositions. In one example, a therapeutically effective amount or concentration is one that is sufficient to prevent advancement, delay progression, or to cause regression of a disease or condition, or which is capable of reducing symptoms caused by any disease or condition.

In one example, a desired effect is to reduce or inhibit one or more symptoms associated with a disease or condition characterized by alphavirus infection. The one or more symptoms do not have to be completely eliminated for the composition to be effective. For example, a composition can decrease the sign or symptom by a desired amount, for example by at least 20%, at least 40%, at least 50%, at least 80%, at least 90%, at least 95%, at least 98%, or even at least 100%, as compared to how the sign or symptom would have progressed in the absence of the composition or in comparison to currently available treatments.

The actual effective amount will vary according to factors such as the type of alphavirus infection to be protected against/therapeutically treated and the particular status of the subject (for example, the subject's age, size, fitness, extent of symptoms, susceptibility factors, and the like) time and route of administration, other drugs or treatments being administered concurrently, as well as the specific pharmacology of treatments for alphavirus infection for eliciting the desired activity or biological response in the subject. Dosage regimens can be adjusted to provide an optimum prophylactic or therapeutic response.

An effective amount is also one in which any toxic or detrimental side effects of the compound and/or other biologically active agent is outweighed in clinical terms by therapeutically beneficial effects. A non-limiting range for a therapeutically effective amount of treatments for alphavirus infection within the methods and formulations of the disclosure is about 0.0001 μg/kg body weight to about 10 mg/kg body weight per dose for one or both compounds in the combination, such as about 0.0001 μg/kg body weight to about 0.001 μg/kg body weight per dose for one or both compounds in the combination, about 0.001 μg/kg body weight to about 0.01 μg/kg body weight per dose for one or both compounds in the combination, about 0.01 μg/kg body weight to about 0.1 μg/kg body weight per dose for one or both compounds in the combination about 0.1 μg/kg body weight to about 10 μg/kg body weight per dose for one or both compounds in the combination, about 1 μg/kg body weight to about 100 μg/kg body weight per dose for one or both compounds in the combination, about 100 μg/kg body weight to about 500 μg/kg body weight per dose for one or both compounds in the combination, about 500 μg/kg body weight per dose to about 1000 μg/kg body weight per dose for one or both compounds in the combination, or about 1.0 mg/kg body weight to about 10 mg/kg body weight per dose for one or both compounds in the combination.

Determination of effective amount is typically based on animal model studies followed up by human clinical trials and is guided by administration protocols that significantly reduce the occurrence or severity of targeted disease or condition symptoms in the subject. Suitable models in this regard include, for example, murine, rat, rabbit, porcine, feline, non-human primate, and other accepted animal model subjects known in the arts. Using such models, only ordinary calculations and adjustments are required to determine an appropriate concentration and dose to administer a therapeutically effective amount of the treatments for hematological malignancies.

### EXAMPLES

### Example 1 Identification of a Novel IFN/IRF3-Inducing Molecule by High Throughput In Vitro Screening

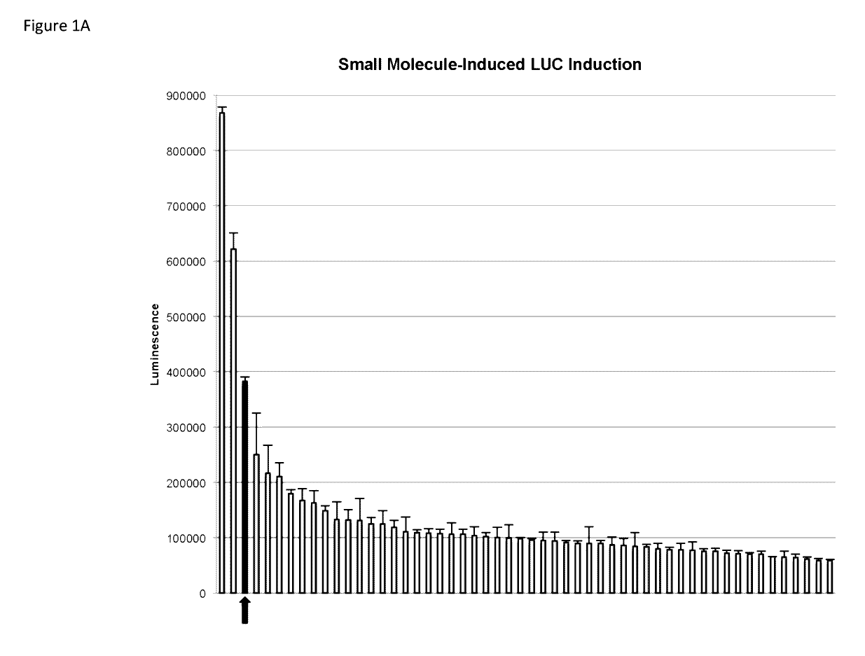

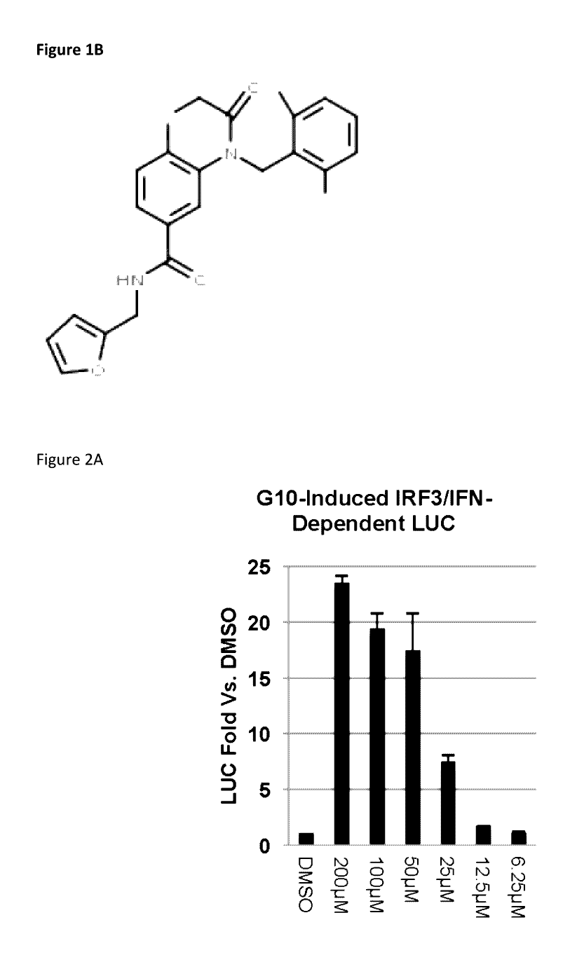

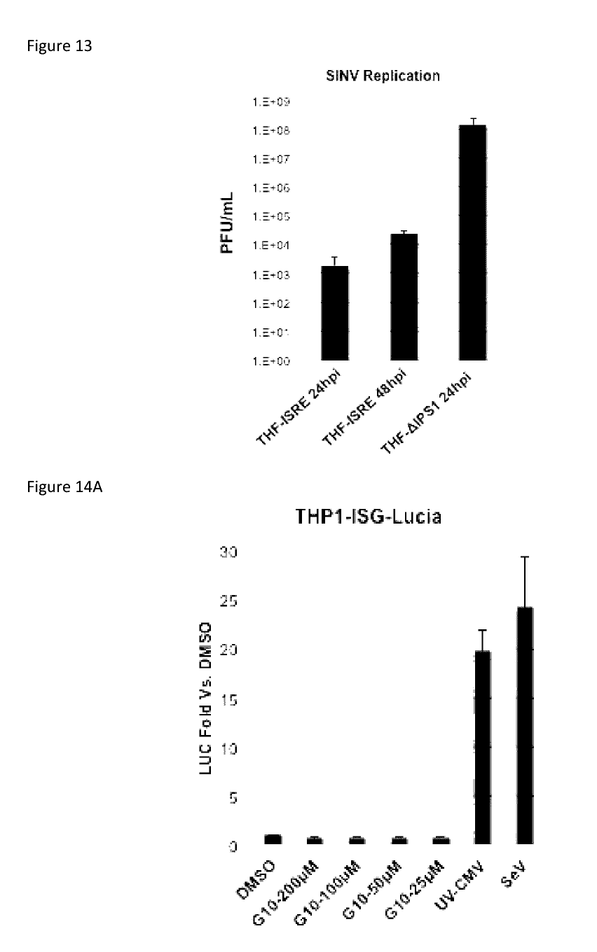

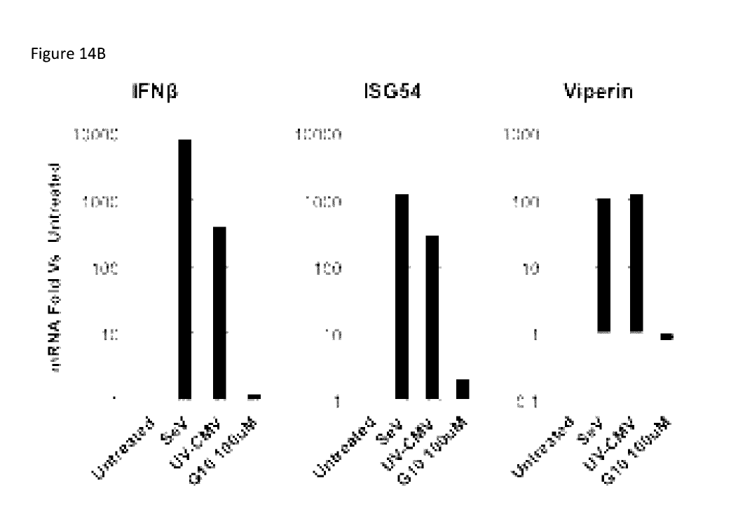

A screening methodology for small molecules capable of stimulating innate immune signaling and effector activity in human cells is disclosed. Human fibroblasts were stably transfected with constitutively expressed human telomerase reverse transcriptase (termed THF) as well as luciferase (LUC) from Phytonis pyralis downstream of a promoter element that is reactive to type I IFN-dependent as well as IRF3-dependent transcription (termed THF-ISRE) [18]. Using these cells in a 384-well high-throughput in vitro screening platform we examined 51,632 chemically diverse compounds in duplicate for their ability to significantly stimulate expression of LUC. Sixteen positive control (1000 U/mL IFNβ) and negative control (1% DMSO) LUC readings were obtained for each plate (μP and μN averages, respectively). Readings for individual compounds (R) on a single plate were designated as significant if R>(μP−μN)*0.5. FIG. 1A illustrates the distribution of raw LUC readings for all molecules that exceeded this threshold on duplicate plates. A compound that exhibited the third highest signal of all those screened was 4-(2-chloro-6-fluorobenzyl)-N-(furan-2-ylmethyl)-3-oxo-3,4-dihydro-2H-benzo[b][1,4]thiazine-6-carboxamide, which we termed G10 (FIG. 1B). As shown in FIG. 13, G10 exhibited low cytotoxicity even at high concentrations and was thus selected for more comprehensive examination.

### Example 2—G10 Induces IFN/IRF3- but not NF-κB-Dependent Transcription in Human Fibroblasts

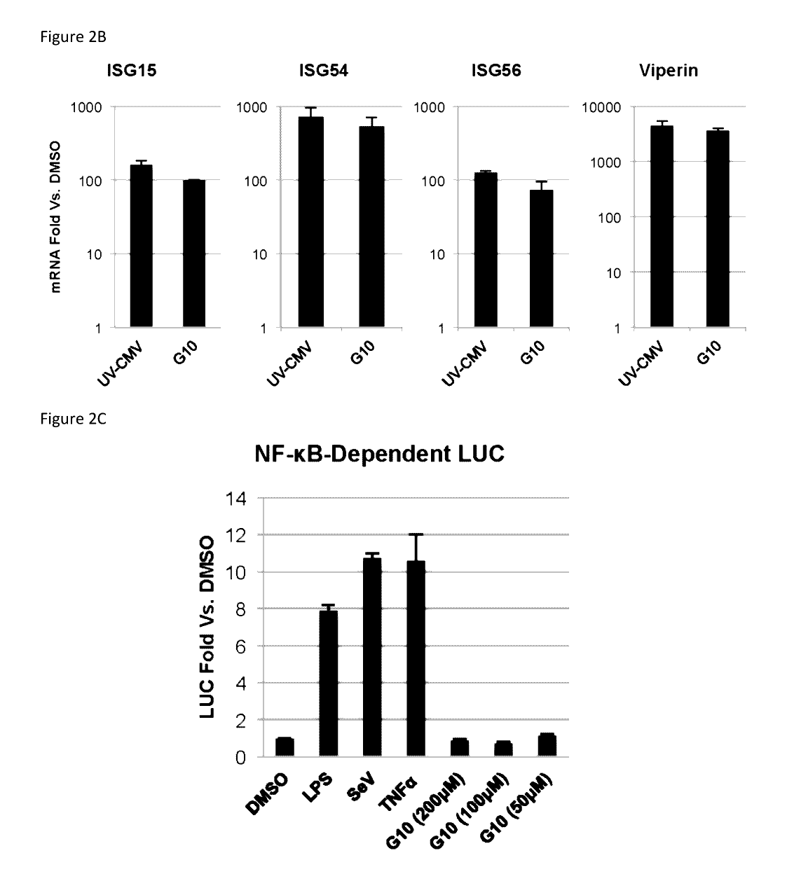

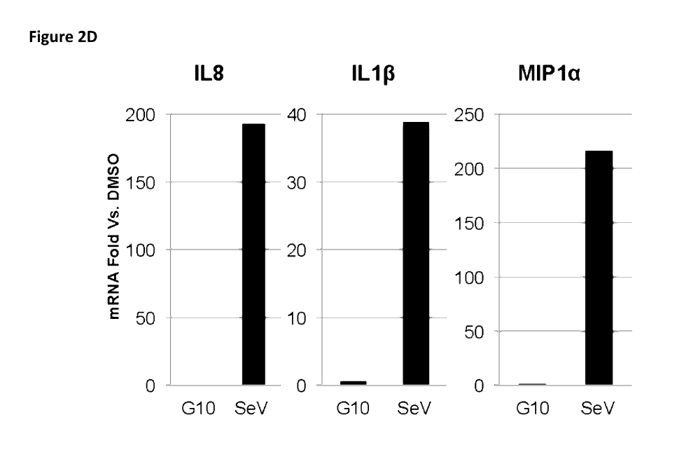

G10-mediated induction of IFN/IRF3-dependent LUC was confirmed by exposing the transfected human fibroblast them to a range of concentrations of G10. As shown in FIG. 2A, the compound was able to induce LUC expression in a dose-dependent manner. Semiquantitative RT-PCR (qPCR) was used to examine the induction of mRNA characterized as dependent on either IRF3 or IFN (ISG54, ISG56, ISG15, Viperin) by G10. As shown in FIG. 2B, exposing cells to G10 led to transcription of cellular genes triggered by IRF3/IFN-dependent signaling in a manner similar to that induced by exposure to human cytomegalovirus rendered replicationally inactive by UV irradiation (UV-CMV); a stimulus of IRF3 and IFN that occurs via STING and ZBP1/DAI (Navarro L et al, Mol Cell Biol 18, 3796-3802 (1998); DeFilippis V R, Mol Cell Biol 80, 1032-1037 (2006); and DeFilippis V R et al, J Virol 84, 8913-8925 (2010); all of which are incorporated by reference herein).

Molecular patterns that culminate in IRF3 activation and synthesis of type I IFN often simultaneously induce pathways leading to activation of the transcription factor NF-κB. It was next examined whether G10 also stimulated this response. Telomerized human fibroblasts (THF) were stably transduced with LUC driven by an NF-κB dependent promoter. As shown in FIG. 2C no G10-associated stimulation of NF-κB-dependent transcription was detected even at the highest concentrations of the compound tested. This contrasts with what is observed in these cells for other NF-κB-inducing stimuli such as Sendai virus (SeV), TNFα, and LPS. Moreover, the compound also failed to stimulate mRNA synthesis of endogenous NF-κB-dependent genes (IL8, IL1β, and MIP1α; FIG. 2D). Overall these results suggest that G10 activates IRF3, but not canonical NF-κB pathways in human fibroblasts.

### Example 3—G10 Elicits Antiviral Activity Against New and Old World Alphaviruses

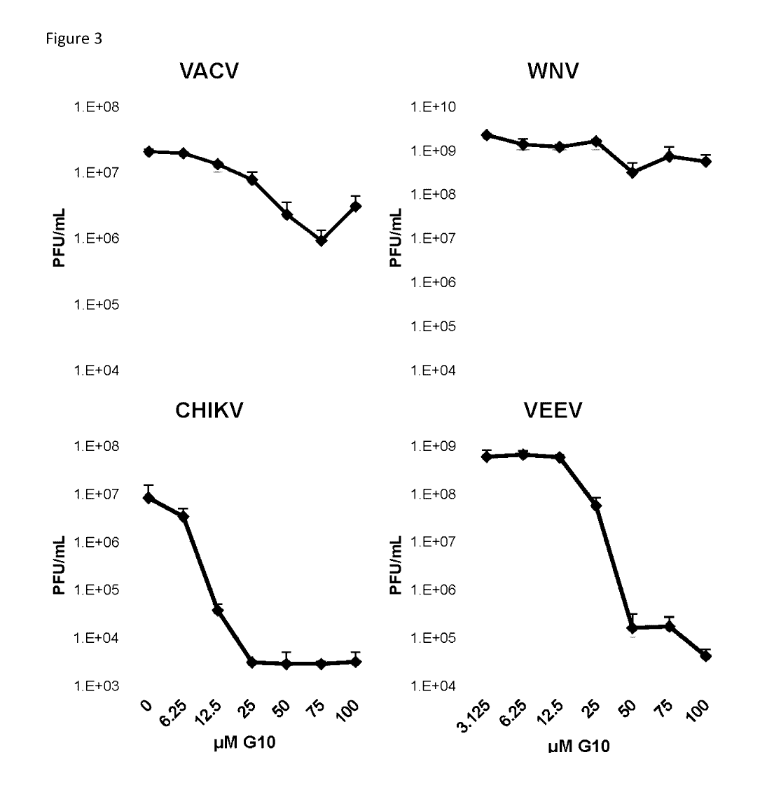

These results indicated that exposure of cells to G10 stimulates the expression of genes that are dependent on IRF3- and/or IFN-dependent signaling. Numerous such genes have been characterized as antiviral effectors that act either via direct molecular or indirect immunological mechanisms (see Schneider W M et al, Ann Rev Immunol 32, 513-545 (2014); incorporated by reference herein). It was therefore examined whether G10 was capable of generating a cellular state refractory to virus replication in vitro, presumably through the initial activity of IRF3. THF cells were pre-exposed for 6 hours to concentrations of G10 that fell well within a nontoxic range (FIG. 13) but were still sufficient to initiate innate gene transcription (FIG. 2). The replication of West Nile Virus (WNV), Vaccinia Virus (VACV), and Chikungunya virus (CHIKV) on these cells was then measured. As shown in FIG. 3, the highest concentration of G10 was only able to reduce growth of VACV by approximately one log and WNV by less than one log. Intriguingly, however, the cellular state induced by G10 was much more inhibitory to the growth of CHIKV, reducing replication by over three logs (IC₉₀=8.01 μM; FIG. 3). In light of this we examined replication of another clinically relevant Alphavirus species Venezuelan Equine Encephalitis Virus (VEEV). As shown in FIG. 3, G10 also potently blocked replication of this virus (IC90=24.57 μM). While related, these species represent highly diverse Alphavirus clades with CHIKV deriving from the Old World and VEEV the New World. Nevertheless G10 was highly effective at similarly impairing replication of both viruses. The relative inability of G10 to render cells as resistant to WNV or VACV replication may reflect the differential susceptibilities of the virus types to the specific antiviral genes induced by G10 or innate evasion phenotypes exhibited by the different viruses (see Maringer K and Fernandez-Selma A, Cytokine Growth Factor Revs 25, 669-679 (2014) and Dai P et al, PLoS Pathol 10, e1003989 (2014); both of which are incorporated by reference herein).

### Example 4—IRF3 is Essential to G10-Induced Transcription and Anti-Alphaviral Activity

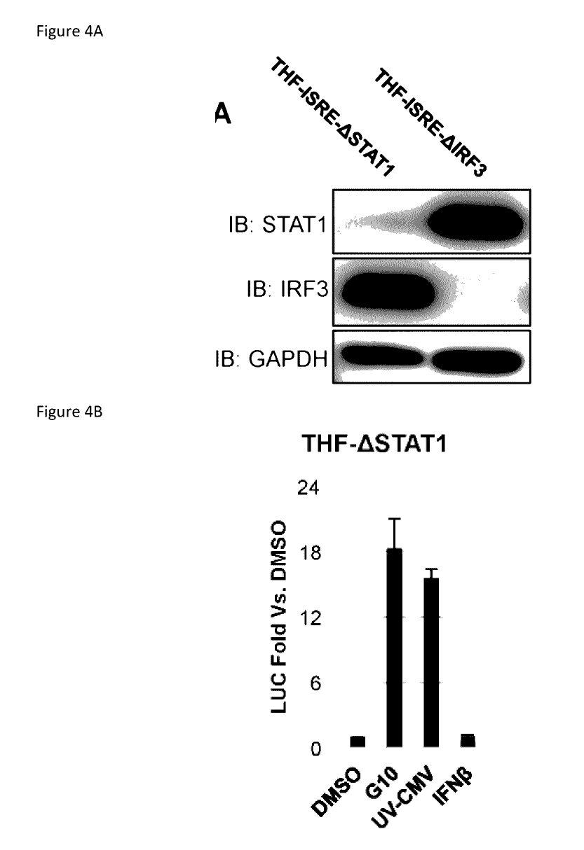

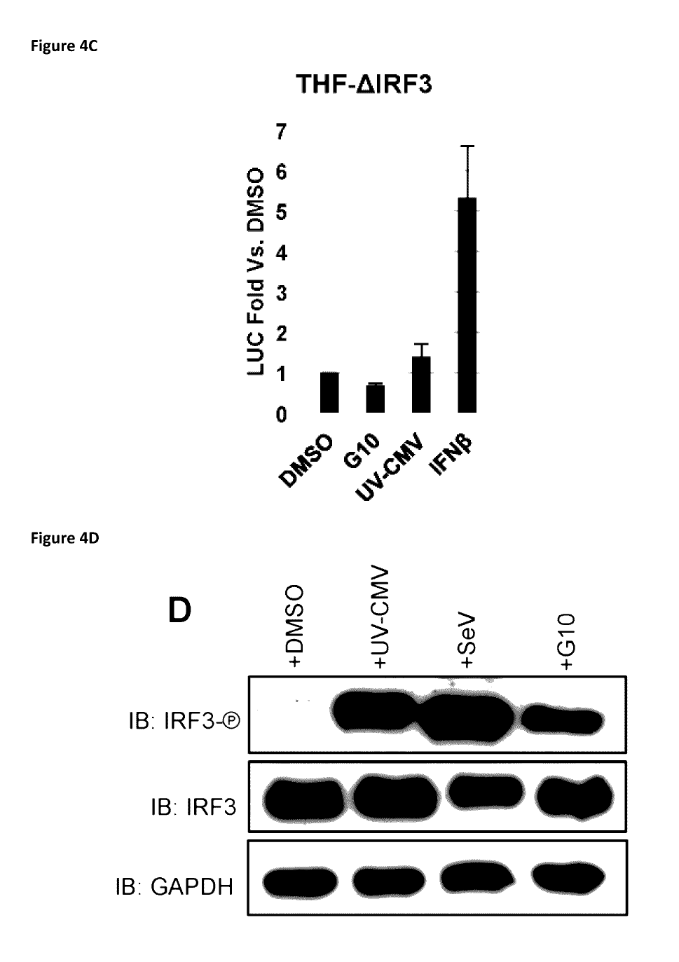

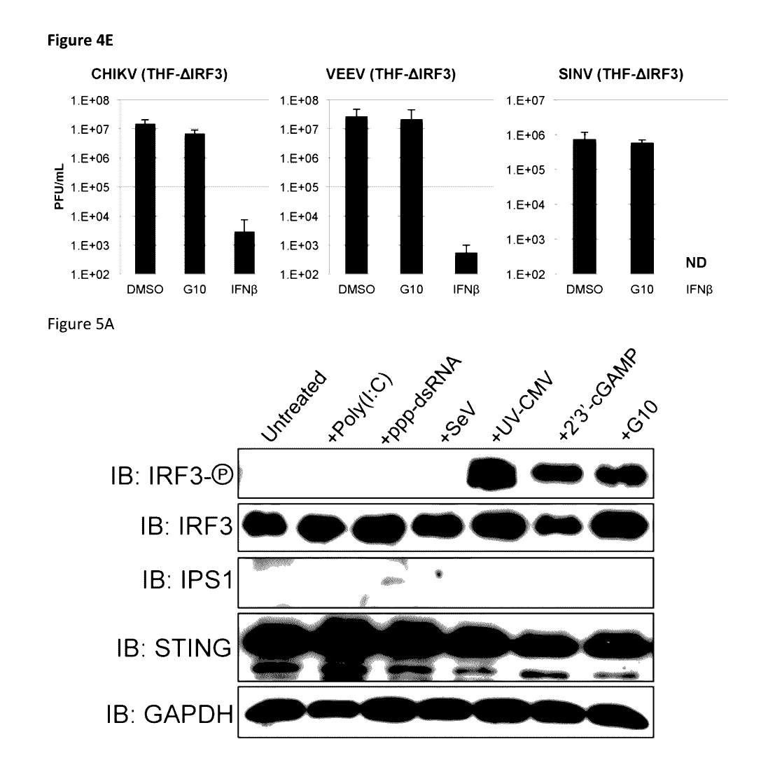

Since LUC expression in THF-ISRE reporter cells can be activated directly by IRF3 alone or following IFN-mediated Jak/STAT1/2 signaling it was next examined whether either or both transcription complexes were required for this effect. Derivative THF-ISRE cells were developed from which either the STAT1 or IRF3 protein was stably removed via disruption of the respective coding regions by lentivirus-delivered CRISPR/Cas9 components (Sanjana N E et al, Nat Meth 11, 783-784 (2014); Sternberg S H et al, RNA 18, 661-672 (2012); Mali P et al, Science 339, 823-826 (2013); and Ran F A et at Nat Protoc 8, 2281-2308 (2013); all of which are incorporated by reference herein). As shown in FIG. 4A cells lacking IRF3 (THF ISRE-ΔIRF3) or STAT1 (THF-ISRE-ΔSTAT1) protein as detectable by immunoblot (IB) were obtained. Absence of these proteins was functionally validated by the elimination of STAT1- (FIG. 4B) or IRF3-dependent (FIG. 4C) LUC expression following treatment with control stimuli IFNβ and UV-inactivated cytomegalovirus (UV-CMV), respectively. Using these cells, G10 was found to induce LUC expression in the absence of STAT1. However, G10-induced LUC expression was abrogated in cells lacking IRF3. These results suggest that the innate immune stimulation observed in response to G10 is dependent on the IRF3 transcription factor and does not require direct activation of Jak/STAT-dependent signaling.

Since transcription of a reporter gene by G10 does not occur in the absence of IRF3 this strongly implies that G10 stimulates the activation of IRF3, which involves phosphorylation of C-terminal serine residues and subsequently allows its dimerization, nuclear translocation and DNA binding. To verify that IRF3 activation does occur in response to G10 IB was performed using an antibody reactive to phosphorylated IRF3 residue S386 with whole cell lysates harvested from THF exposed to G10 or control stimuli. As shown in FIG. 4D G10 stimulates phosphorylation of IRF3 in a manner similar to that triggered by UV-CMV and SeV. It was next examined whether IRF3 was involved in establishment of the observed G10-mediated anti-Alphaviral state (FIG. 3). To answer this THF-ISRE-ΔIRF3 were exposed to DMSO, 100 μM G10 (a concentration over twelve times the IC₉₀ for CHIKV and over 264 four times the IC₉₀ for VEEV), or 1000 U/mL IFNβ and examined growth of CHIKV and VEEV after a period that enables peak viral titers. FIG. 4E shows that while a strong anti-Alphaviral state can still be established in these cells by pre-exposure to IFNβ, the ability of G10 to block replication of CHIKV and VEEV is lost. In addition, Sindbis virus (SINV), another Old World Alphavirus species, that grows poorly on wild type human fibroblasts was also examined (FIG. 13) but can replicate when the IPS-1-IRF3-IFN response is impaired. As shown in FIG. 4E cells lacking IRF3 are permissive for SINV replication. However, in these cells IFNβ, but not G10, is capable of inhibiting virus replication. These results indicate the anti-Alphaviral activity elicited by G10 requires IRF3-dependent cellular responses.

### Example 5—G10-Mediated IRF3 Activation and Anti-Alphaviral Activity Occur Independently of IPS-1/MAVS-Dependent Signaling

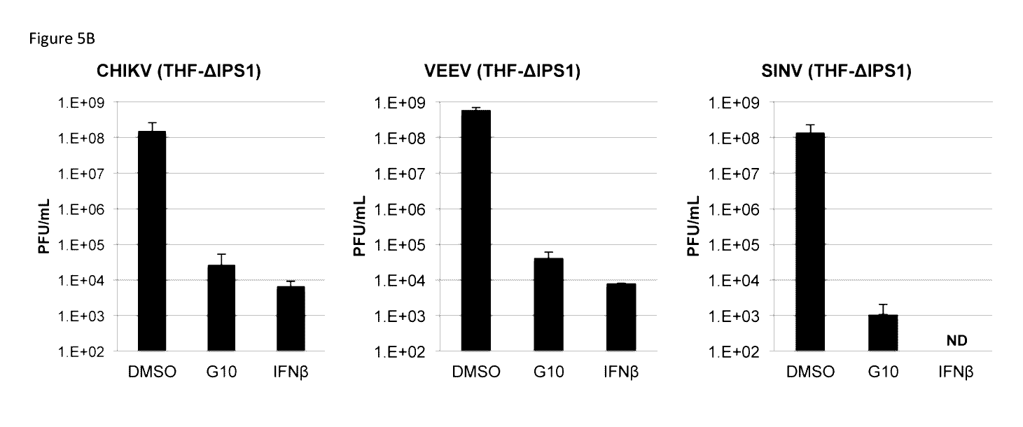

An array of PRRs reacting with multiple classes of pathogen-associated molecules is capable of initiating signaling pathways that terminate in IRF3 activation. As discussed above these conventionally employ an adaptor protein to activate the IRF3-directed kinases TBK1 and IKKε. IFNβ promoter stimulator 1 (IPS-1, also called MAVS) is utilized by RIG-I and MDA5, cytoplasmic sensors of (typically virus-associated) dsRNA. In an effort to characterize the cellular pathway targeted by G10 it was first asked whether IPS-1 is important for the molecule's effect on innate cellular activation. Lentivirus-delivered CRISPR/Cas9 was used to construct THF cells lacking the protein. As shown in FIG. 5A, disruption of the IPS-1 coding region correspondingly results in undetectable protein. Furthermore, IRF3 activation in response to poly(I:C), ppp-dsRNA, or SeV infection, processes requiring IPS-1 (Hornung V et al, Science 314, 994-997 (2006) and Gitlin L et al, Proc Natl Acad Sci 103, 8459-8464 (2006); both of which are incorporated by reference herein), are accordingly abrogated in these cells. In contrast, IRF3 activation by UV-CMV or 2′3′-cGAMP, which occur via the STING pathway in these cells (Ablasser A et al, Nature 498, 380-384 (2013); incorporated by reference herein) and are independent of IPS-1 remains intact. Likewise, G10-induced IRF3 phosphorylation is also functional in these cells (FIG. 5A) indicating that the molecule stimulates a response that does not require components of the IPS-1 signaling apparatus. It was then asked whether the anti-Alphaviral activity elicited by G10 similarly occurred independently of IPS-1 as described above. As shown in FIG. 5B, pretreatment of cells with either G10 or IFNβ diminishes replication of all three virus types by multiple logs; a magnitude that parallels what is observed in wild type cells (FIG. 3). Together these results indicate that the IRF3-dependent anti-Alphaviral activity conferred by G10 does not require IPS-1 or, by extension, any upstream PRRs (i.e. RIG-I, MDA5, POL3) associated with this signaling pathway.

### Example 6—STING is Required for G10-Mediated IRF3 Activation, Gene Expression, and Anti-Alphaviral Activity

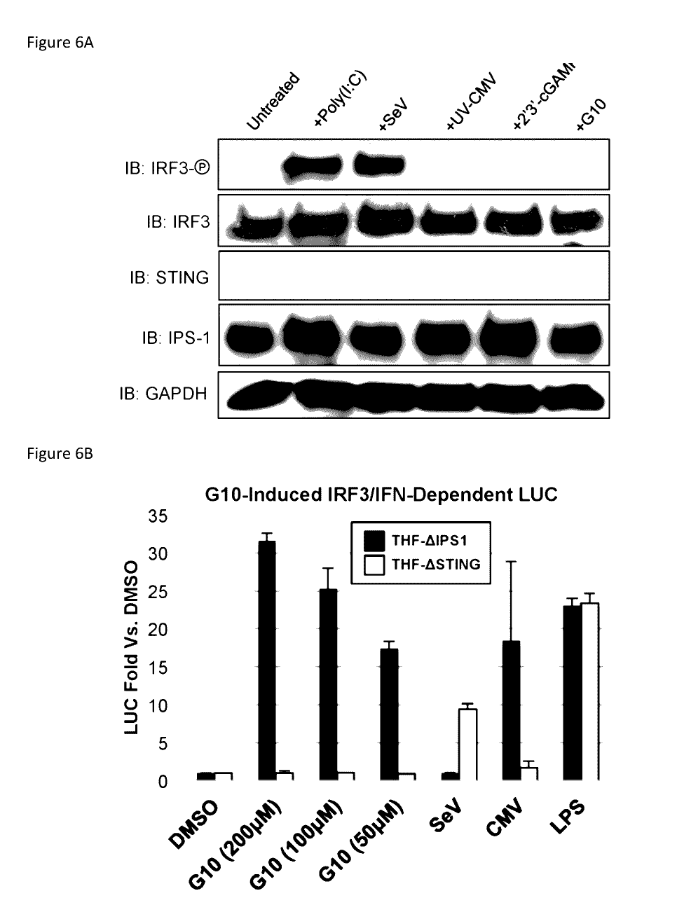

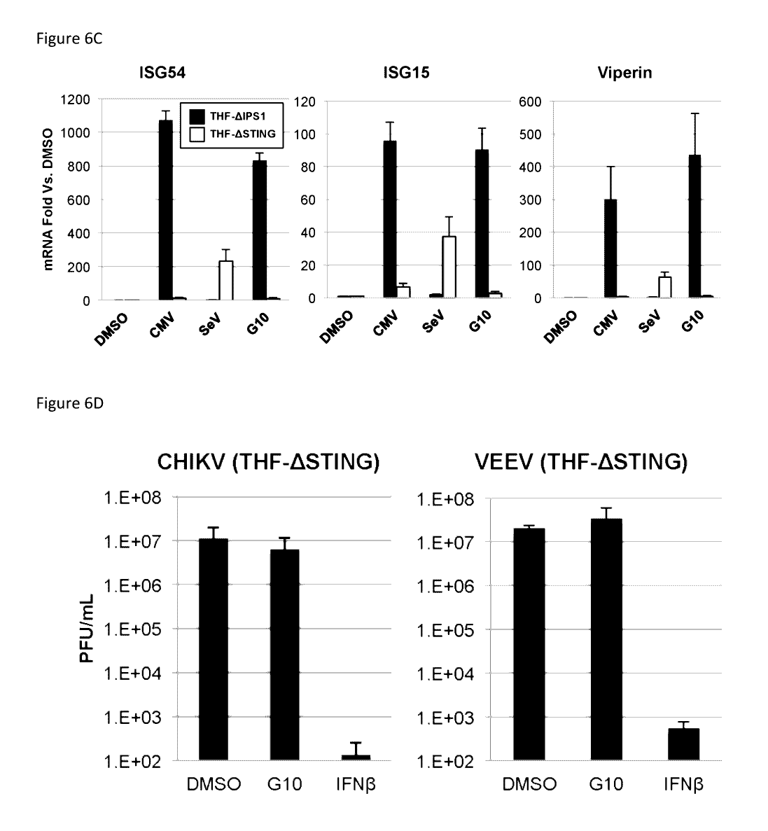

It was then examined whether the IRF3-terminal adaptor protein STING is critical to G10 mediated innate activation. THF-ISRE cells were constructed from which the STING protein is eliminated via CRISPR/Cas9-mediated gene disruption as described above. Knockout of the protein was confirmed visually by IB of whole cell lysates and functionally by demonstrating the absence of IRF3 S386 phosphorylation following treatment with UV-CMV or transfection with 2′3′-cGAMP, both of which are STING dependent cellular reactions (FIG. 6A). IPS-1-dependent signaling remains operational in these cells, however, as evidenced by IRF3 phosphorylation in response to SeV exposure and poly(I:C) transfection. Intriguingly, STING deletion resulted in elimination of G10-induced IRF3 S386 phosphorylation. It was next examined whether the G10-induced transcriptional response observed in wild type cells (FIG. 2) was similarly inactive in STING-deficient cells. For this we also included THF-ISRE-ΔIPS-1 cells as a control for any potential off-target effects of CRISPR/Cas9 genome editing or lentivirus transduction. As shown in FIG. 6B, treatment with G10 leads to strong LUC induction in cells lacking IPS-1, as does exposure to UV-CMV or LPS (a TRIF-dependent process) but not SeV. However, in the absence of STING both G10 and UV-CMV fail to activate appreciable LUC whereas SeV and LPS induce substantial expression thus validating intact IRF3-terminal signaling. Transcriptional induction of IRF3-dependent endogenous host genes was also examined by qPCR. FIG. 6C shows that, similar to LUC expression, ISG54, ISG15, and Viperin mRNAs are highly transcribed in response to UV-CMV and G10 exposure in cells lacking IPS-1 but remain comparatively unstimulated in cells lacking STING. In contrast, SeV-induced transcription of these genes is only observed in cells lacking STING. From these results it can be concluded that the IRF3 activation and IRF3-dependent transcriptional activity in response to G10 occurs via a STING-dependent pathway.

Based on our observations above that G10-elicited anti-Alphaviral activity requires IRF3 (FIG. 4), it was hypothesized that STING-dependent IRF3 signaling is also essential to this process. Replication of CHIKV and VEEV in THF-ISRE-ΔSTING cells following treatment with DMSO, G10, or IFNβ was examined as described above (these cells are not permissive for SINV replication since IPS-1-IRF3-IFN signaling is intact (White L K et al, J Virol 85, 606-620 (2011); incorporated by reference herein). As shown in FIG. 6D the ability of G10 to block virus replication is absent in cells lacking STING despite the fact that an antiviral state can effectively be established as indicated by treatment with IFNβ. Overall these data clearly indicate that the STING pathway is crucial to the IRF3-dependent innate antiviral activity induced by G10.

STING behaves as a PRR of cyclic dinucleotides (CDN) by way of a direct interaction between them and the protein's C-terminal (ligand-binding) domain (Burdette D L and Vance R E, Nat Immunol 14, 19-26 (2013); incorporated by reference herein). It was therefore asked whether G10 was a direct ligand of human STING, similar to the mouse STING345 specific small molecules DMXAA (Gao P et al, Cell 154, 748-762 (2013) and Prantner D et al, J Biol Chem 287, 39776-39788 (2012); both of which are incorporated by reference herein) and CMA, and sought evidence supporting this hypothesis. Differential scanning fluorimetry was used to examine changes in thermal stability of purified STING-CTD in the presence of G10. Thermal stability of the protein is expected to increase with binding affinity of protein-ligand complexes (Niesen F H et al, Nat Protoc 2, 2212-2221 (2007) and Zhang X et al, 51, 226-235 (2013); both of which are incorporated by reference herein). However, as shown in FIG. 14, incubating purified mouse or human STING-CTD with G10 did not increase the protein's thermal stability, as does a validated ligand such as 2′3′-cGAMP or DMXAA (in the case of mouse protein). These observations are inconsistent with G10 binding directly to human or mouse STING-CTD.

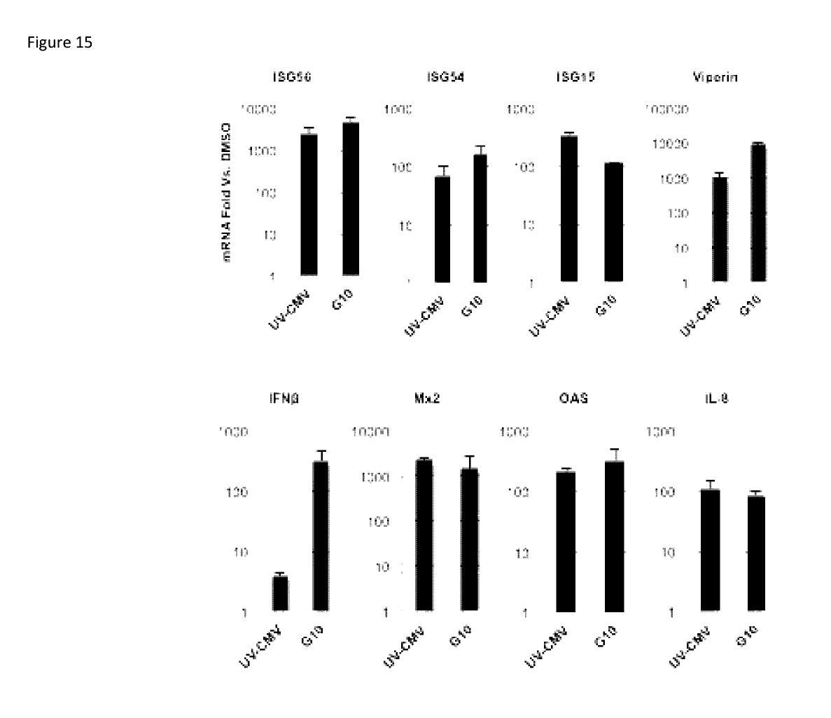

STING also behaves as an adaptor molecule [68] required for activating IRF3-targeting kinases by multiple upstream cytoplasmic DNA-sensing PRRs including ZBP1/DAI (DeFilippis V R et al, J Virol 84, 585-598 (2010); incorporated by reference herein), IFI16, DDX41 (Parvatiyar K et al, Nat Immunol 13, 1155-1161 (2012); incorporated by reference herein and IFI203. Given that evidence of direct interaction was not found with STING it is possible that G10 engages one or more of these (or an as yet unknown; see Motani K et al, J Immunol 194, 4914-4923 (2015); incorporated by reference herein) PRRs to initiate STING-dependent activity. In light of this it is interesting to note that G10 does not induce IRF3 activation or IRF3-dependent gene expression in the immortalized promonocytic cell line THP-1 despite the fact that these cells express phenotypically active STING (Li Z et al, PLoS Pathol 11, e1004783-26 (2015); Zhang Z et al, Nat Immunol 12, 959-965 (2011); Jakobsen M R et al, Proc Natl Acad Sci USA 110, E4571-E4580 (2013); Mankan A K et al, EMBO J 33, 2937-2946 (2014); Sun C et al, J Immunol 194, 1819-1831 (2015); and Panchanathan R et al, Innate Immunity 20, 751-759 (2014); all of which are incorporated by reference herein) (FIG. 15). These results are potentially informative with respect to identity of the direct cellular target of G10 since it likely rules out cGAS, DDX41, and IFI16 based on the observation that these are present and functional in THP-1 cells (Hansen K et al, EMBO J 33, 1654-1666 (2014); incorporated by reference herein). Without being bound by theory, it is possible that known STING-dependent receptors (IFI203 and ZBP1/DAI) are required for G10-mediated activity.

### Example 7—G10 Induces STING-Dependent Synthesis and Secretion of Bioactive Interferon

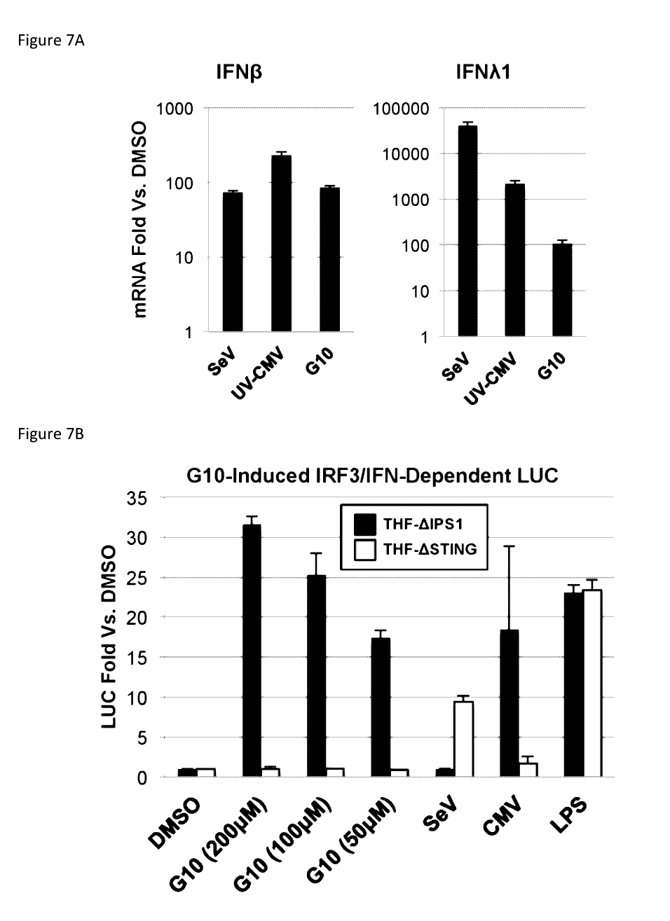

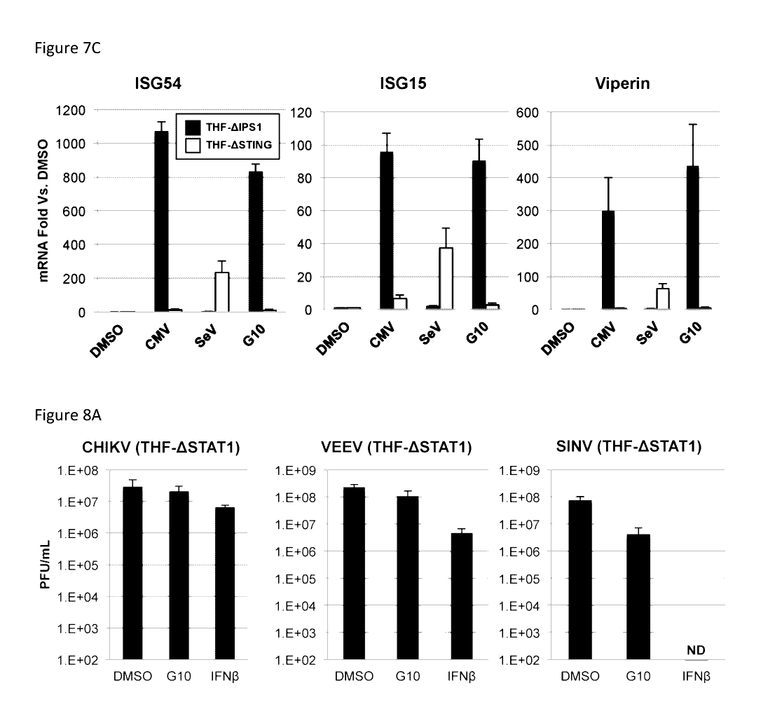

Our data indicate that G10 induces expression of cellular antiviral effector genes and that this process ultimately requires IRF3 and STING. However, transcription of these genes (ISG15, Viperin, ISG54, ISG56) can be triggered in response to either activated IRF3 or IFN-dependent (Jak/STAT) signaling (Grandvaux X et al, J Virol 76, 5532-5539 (2002); Elco C P et al, J Virol 79, 3920-3929 (2005); and Noyce R S et al, J Virol 80, 226-235 (2006); all of which are incorporated by reference herein). It was first examined whether G10 is able to stimulate expression of type I or III interferons, both of which are known to induce Jak/STAT-dependent signaling via type I and type III IFN receptor complexes, respectively. As shown in FIG. 7A, exposure of THF-ISRE to G10 leads to transcription of both IFNβ and IFNλ1 mRNAs. However, induction of IFNλ1 by G10 does not approach the levels observed for UV-CMV and SeV suggesting the involvement of different, potentially G10-independent, cellular factors in this process. It was next examined whether genes known to be induced solely by Jak/STAT- (as opposed to either Jak/STAT or IRF3) dependent signaling were also induced following G10 exposure since this would be indicative of autocrine/paracrine signaling following release of type I or type III IFN from treated cells. In contrast to the 7 h treatments used above (FIGS. 2 and 6) cells were exposed to the indicated stimuli for 18 hours in order to allow for completion of the full sequence of IFN transcription, synthesis, secretion, and autocrine/paracrine signal transduction. FIG. 7B illustrates substantial induction of known IFNα/β-dependent genes Mx2 and OAS in response to exposure to G10 as well as other control stimuli including IFNβ. These results are consistent with the secretion of IFN in response to treatment with G10.

Numerous subtypes of type I and III interferons exist 393 and thus demonstrating the presence of secreted molecules requires type-specific immunoassays (ELISA). Secreted interferon of all subtypes was examined using a cell-based reporter assay that reacts with any bioactive type I or III interferon species. For this we utilized THF-ISRE-ΔIRF3 cells described above (FIG. 4). Since these cells cannot make type I IFN (due to the absence of IRF3) they do not generate an autocrine LUC reporter signal in response to exogenous IFN-inducing stimuli and only react to IFN itself (FIG. 4). THF-ISRE, THF-ISRE-ΔIPS-1, and THF-ISRE-ΔSTING were treated with DMSO, SeV, UV CMV, or G10 for 18 hours and transferred media from these cells to THF-ISRE-ΔIRF3 for 8 h. Next, IFN-dependent luciferase expression was measured by luminescence. In agreement with our previous observations, IFN secretion was detected by wild type THF cells exposed to UV-CMV, SeV, or G10. In cells lacking IPS-1 secretion of IFN was abolished in response to SeV but not UV-CMV or G10. Moreover, cells lacking STING failed to secrete IFN in response to UV-CMV or G10 but SeV-induced secretion was intact. Interestingly, G10-induced secretion by cells lacking IPS-1 was significantly diminished relative to wild type cells (p=0.00013) perhaps indicating pivotal crosstalk between STING and IPS-1 signaling. However, as indicated by FIG. 6 the role of IPS-1 does not appear to be essential for G10's anti-Alphaviral activity. Overall these data indicate that G10 triggers the STING dependent transcription, synthesis, and secretion of IFN species capable of initiating Jak/STAT signaling and ISG expression.

### Example 8—STAT1 is Required for G10-Mediated Anti-Alphaviral Activity and ISG Expression

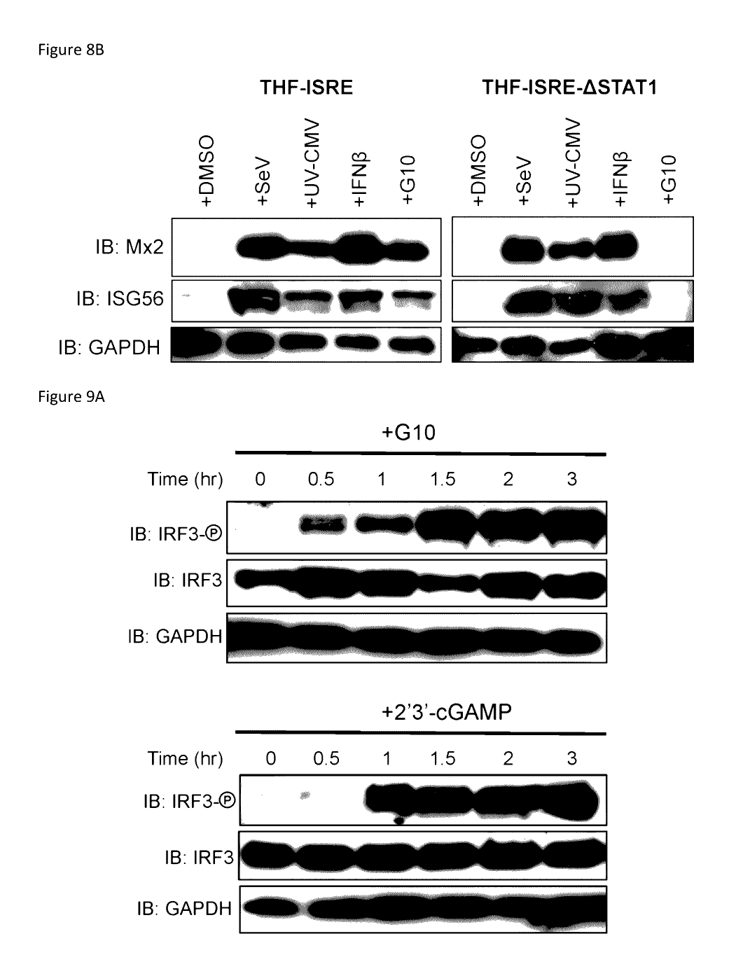

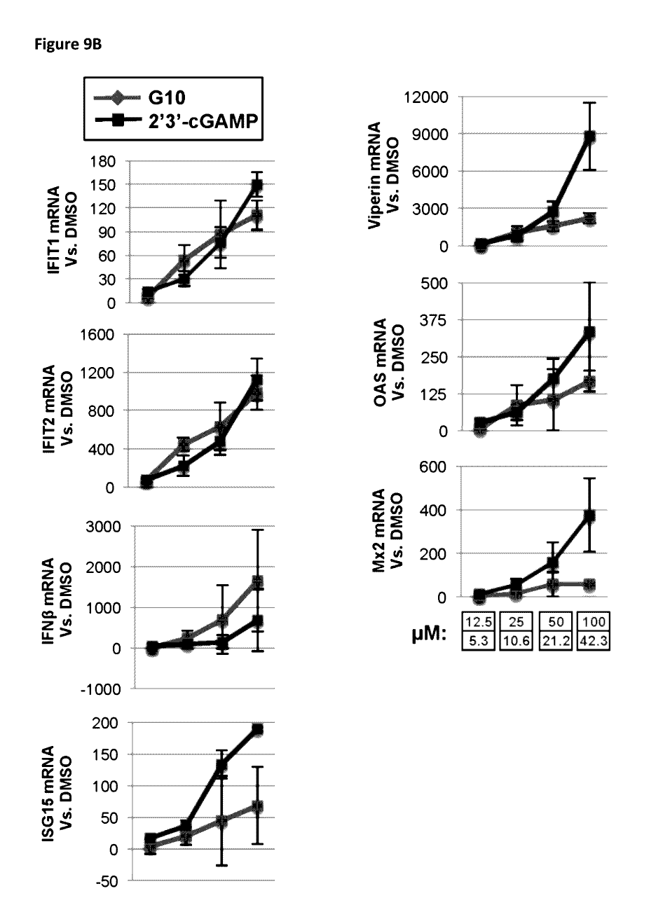

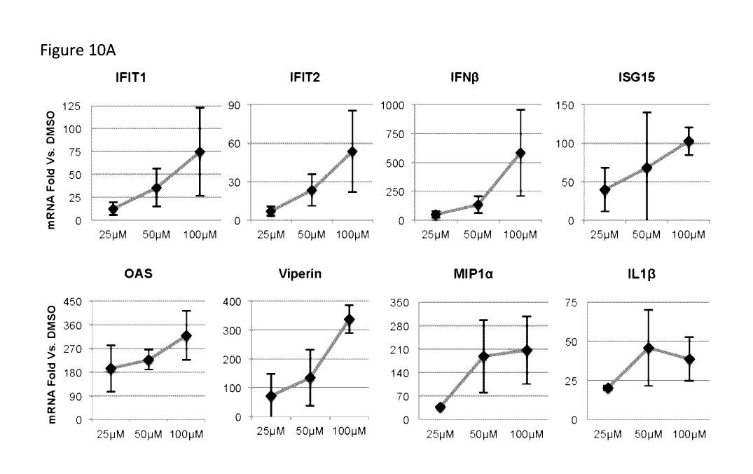

G10 exposure elicits secretion of bioactive IFN in cells that contain STING and IRF3 (FIG. 7C) but also the expression of two classes of ISGs: Those that are induced by either IRF3- or IFN-dependent signaling (ISG15, ISG54, ISG56, Viperin) and those whose expression is activated only by IFN-mediated Jak/STAT signaling (Mx2, OAS). Since many IRF3-dependent genes are direct antiviral effectors it was then asked whether the G10-mediated anti-Alphaviral cellular state could be elicited in the absence of canonical STAT1-mediated, IFNα/β-induced signaling by examining replication of CHIKV, VEEV, and SINV on cells lacking STAT1. As shown in FIG. 8A replication of these viruses in cells treated with G10 from which STAT1 was deleted is similar to that seen in the presence of DMSO alone. Intriguingly, IFNβ appears to induce detectable yet significant (in the case of VEEV and CHIKV) and profound (in the case of SINV) antiviral effects even in the absence of STAT1. These data suggest the expression of antiviral effectors that are IFN-induced yet independent of STAT1. Cells lacking STAT1 can still react to IFN-exposure via the activity of STAT2, STAT3, STAT4, or STAT5. In fact, expression of antiviral ISGs (including those examined here) has been detected in the absence of STAT1, likely via STAT2 homodimers (Abdul-Sater A A et al, J Immunol 195, 210-216 (2015); Blaszczyk K et al, Biochem J 466, 511-524 (2015); and Sarkis P T N et al, J Immunol 177, 4530-4540 (2006); all of which are incorporated by reference herein). In light of these results it was examined whether IFN or G10 could stimulate expression of either class of ISG in the absence of STAT1. As shown in FIG. 8B SeV, UV-CMV, IFNβ, and G10 were all capable of triggering synthesis of Mx2 and ISG56 proteins. Surprisingly, however, in STAT1-deficient cells Mx2 and ISG56 proteins were still detectable following exposure to SeV, UV-CMV, and IFNβ but not G10. These results are consistent with previous studies showing that some IFN-responsive antiviral effectors are inducible in the absence of STAT1 (Hahm B et al, Immunity 22, 247-257 (2005) and Ousman S S et al, J Virol 79, 7514-7527 (2005); both of which are incorporated by reference herein).

### Example 9—G10 Triggers IRF3-Dependent Activity with Less Potency than 2′3′-cGAMP

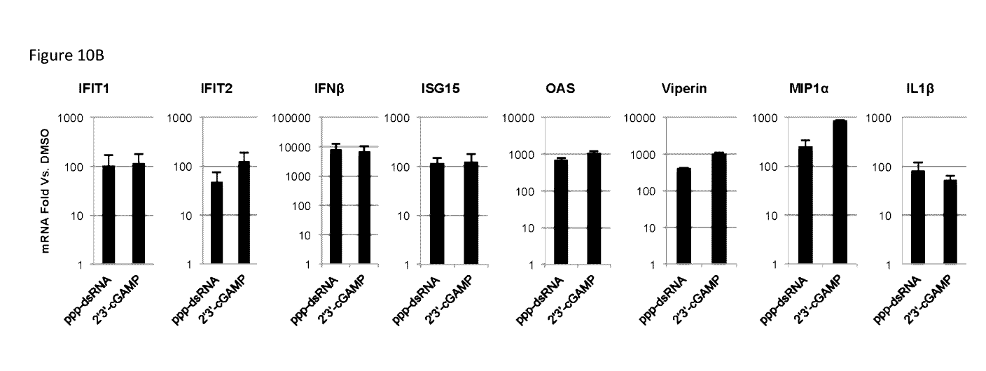

Our observations indicating that STING is required for G10-mediated induction of IRF3-dependent cellular activity led us to examine how the response to the molecule compares to that of a canonical STING ligand such as 2′3′-cGAMP. For this the kinetics of IRF3 phosphorylation and levels of IRF3-dependent gene induction were examined following exposure to each of the molecules. As shown in FIG. 9A phosphorylation of IRF3 S386 is detectable at 30 m post exposure to G10 and at 1 h post exposure to 2′3′-cGAMP. It is important to note that 2′3′-cGAMP was transfected into THF and this may affect timing of observable IRF3 phosphorylation. The transcriptional induction of IRF3- and IFN-dependent genes in response to a range of G10 and 2′3′-cGAMP concentrations was next examined. FIG. 9B illustrates that 2′3′-cGAMP elicits stronger mRNA synthesis of IFIT1, IFIT2, ISG15, Viperin, OAS, and Mx2 at lower concentrations than G10. Oddly, transcription of IFNβ appeared to show a somewhat different pattern with dose-dependent induction being roughly similar between the molecules. These results indicate that innate immune reactivity may occur more quickly in cells exposed directly to G10 relative to 2′3′-cGAMP (following transfection) but that higher concentrations of the molecule are needed to trigger similar levels of IRF3/IFN459 dependent gene expression.

### Example 10—G10 Induces Innate Antiviral mRNA Expression in Primary Human Cells

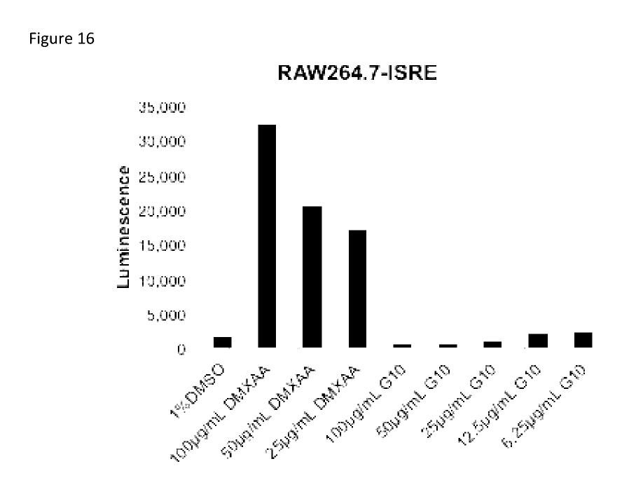

To this point our examination of G10-mediated innate activation has focused on human fibroblasts that, while not strictly immortalized, are life-extended through the introduction of telomerase reverse transcriptase. To determine whether G10 is immunostimulatory to a similar degree in more physiologically relevant primary cell types the transcription of IRF3-, IFN-, and NF-κB-dependent genes in human peripheral blood mononuclear cells (PBMCs) was examined. As shown in FIG. 10, G10 triggered expression of IFIT1, IFIT2, IFNβ, ISG15, OAS, and Viperin to degrees that were proportional to compound concentration. Interestingly, NF-κB-dependent genes MIP1α and IL1β were also transcriptionally induced by G10, a phenomenon not observed in THF cells (FIG. 2). Respective IPS-1- and STING-inducing control stimuli ppp-dsRNA and 2′3′-cGAMP were similarly capable of activating transcription of these genes in these cells (FIG. 10B). As shown in FIG. 16 primary human umbilical microvascular endothelial cells also responded to G10 exposure by expressing IRF3-, IFN-, and NF-κB-dependent genes. These results clearly demonstrate that innate immune induction by G10 is not an effect specific to the cell model and suggests that in vivo stimulation by G10 is likely to be feasible.

### Example 11—STING Agonism Inhibits CHIKV Replication In Vivo

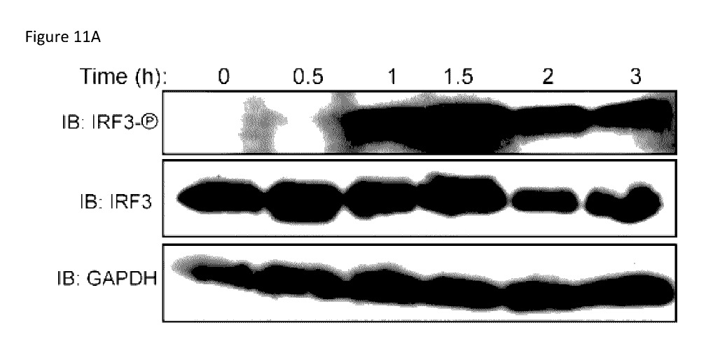

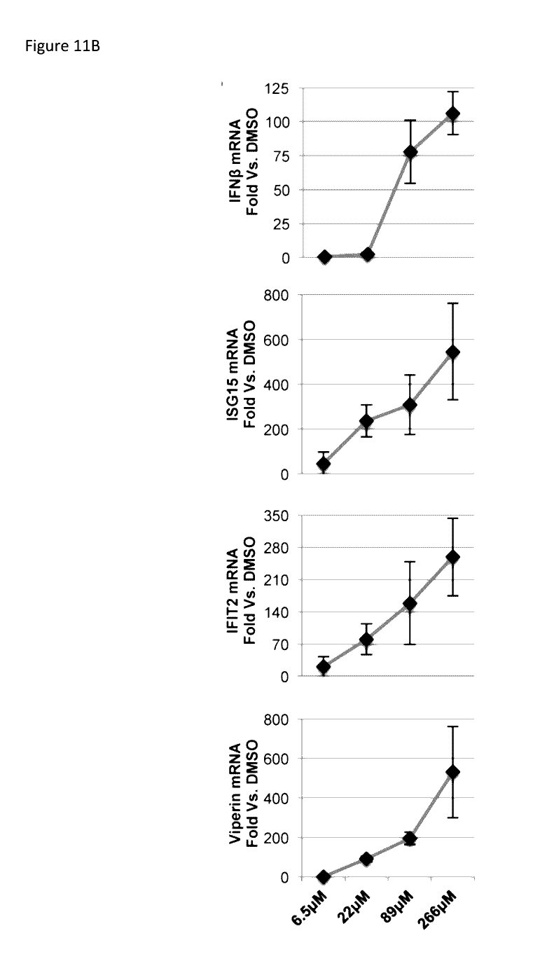

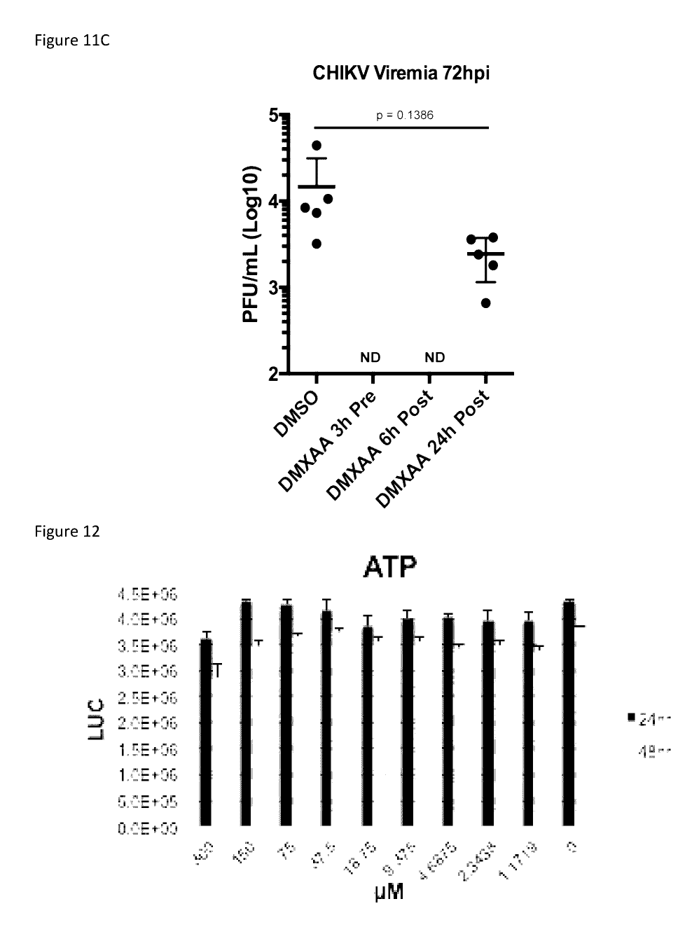

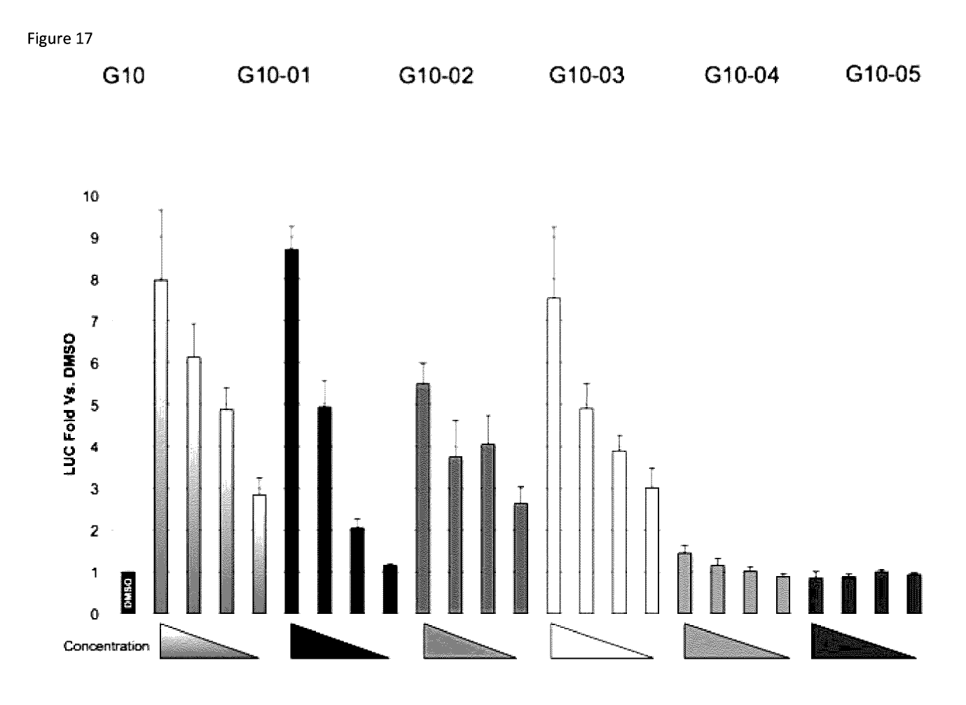

While G10-induced innate immune activation is observed in multiple human cell types, similar activity was not detected in murine myeloid-derived RAW264.7 cells. Multiple molecular analogs of G10 were thus constructed in an attempt to identify one that is active in both human and mouse cells. While this allowed characterization of essential and nonessential moieties within the molecule (FIG. 17), no derivatives were identified that were active across species. However, 5,6-dimethylxanthenone-4-acetic acid (DMXAA) is a small molecule that has been examined extensively and shown to trigger STING-dependent IRF3 and interferon activity in murine but not human cells (FIGS. 14 and 17). As such, the compound has been explored for multiple immunotherapeutic uses including anti-angiogenesis (Cao Z et al, Cancer Res 61, 1517-1521 (2001); incorporated by reference herein), vaccine adjuvanticity (Tang C K et al, PLoS ONE 8, e60038 (2013); incorporated by reference herein), anti-tumor immunology and antiviral activity (Guo F et al, Antimicrob Agents Chemother 59, 1273-1281 (2015); incorporated by reference herein). Patterns of DMXAA-stimulated innate immune activation in murine cells were then explored to evaluate their resemblance with those induced by G10 in human cells. FIG. 11A illustrates that DMXAA-induced IRF3 phosphorylation in RAW264.7 macrophage-like cells is detectable by 1 h post-treatment. Furthermore, DMXAA also elicits dose-dependent transcription of key innate antiviral genes IFNβ, ISG15, IFIT2, and Viperin in a manner similar to that observed for G10 in human cells (FIG. 11B). The physiological effects of DMXAA thus appear to resemble those observed for G10. Fortunately, wild type mice represent a commonly used model of CHIKV infection that manifests viremia, pathogenesis, and immune responses resembling those seen in human patients (Ziegler S A et al, Am J Trop Med Hyg 79, 133-139 (2008); Gardner J et al, J Virol 84, 8021-8032 (2010); Morrison T E et al, Am J Pathol 178, 32-40 (2011); all of which are incorporated by reference herein). It was therefore decided to use this model to ask whether artificial stimulation of STING-dependent signaling was sufficient to block CHIKV replication in vivo.

DMSO or DMXAA (25 mg/kg) were administered to mice intraperitoneally at 3 h pre-infection with CHIKV (1000 PFU). As shown in FIG. 11 treatment with DMSO resulted in an average titer of 1.47×10⁴ PFU/mL serum at 72 h post infection. In contrast, serum associated virus was undetectable by plaque assay in mice pre-treated with DMXAA. It was next examined whether viral titers were influenced by DMXAA given post inoculation. For this DMXAA was administered at 6 h and 24 h post CHIKV infection. FIG. 11 illustrates that virus was still undetectable in serum from mice treated at 6 h post infection. However, mice treated at 24 h post infection showed viral titers that were diminished relative to DMSO-treated animals but this difference was not statistically significant (p=0.1386). Overall these data indicate that artificial stimulation of STING dependent signaling in vivo within an appropriate temporal window can block CHIKV replication. It is likely that the antiviral efficacy of STING activation will diminish as time after acute viral replication increases and viral innate evasion phenotypes appear. It is also possible that recurrent STING activation could have an effect on persistence of CHIKV genomic RNA in joint tissues (Hawman D W et al, J Virol 87, 13878-13888 (2013); incorporated by reference herein).

Pharmacologic activation of STING-dependent signaling represents a potentially high impact therapeutic strategy with applications in diverse clinical areas such as broad spectrum antivirals, vaccine adjuvants, vascular disruption, and antitumor immunology. This is represented by multiple successes of the utilization of this approach in mouse models of virus infection (Taylor J L et al, J Infect Dis 142, 394-399 (1980); incorporated by reference herein), enhancement of vaccine immunogenicity (Dubensky T W et al, Ther Adv Vaccines 1, 131-143 (2013); Li X D et al, Science 341, 1390-1394 (2013); and Hanson M C et al, J Clin Invest 125, 2532-2546 (2015); all of which are incorporated by reference herein), immune-mediated tumor necrosis (Peng S et al, J Biomed Sci 18, 21 (2011); incorporated by reference herein), and inhibition of solid tumor angiogenesis (Ching L M et al, Br J Cancer 86, 1937-1942 (2002); Baguley B C and Ching L M, Intl Radiat Oncol Biol Phys 54, 1503-1511 (2002); both of which are incorporated by reference herein). Unfortunately, synthetic small molecules identified thus far have only exhibited suitable efficacy in mouse models due to their strict specificity for the murine STING ortholog. High-throughput screening was used to identify a novel compound (G10) capable of triggering IRF3/IFN-dependent responses and subsequently blocking replication of CHIKV, VEEV, and SINV in human cells. Follow-up work seeking to pinpoint cellular targets essential to the phenotypic responses utilized a reverse genetics approach by way of CRISPR/Cas9-mediated genome editing. This enabled identification of the STING protein as required for G10's biological activity thus indicating that the compound is the first described human-specific synthetic small molecule STING agonist.

G10 triggers innate immune responses that involve expression of IRF3-dependent genes including type I and III interferons. This was observed in telomerized foreskin fibroblasts as well as primary cells such as PBMCs and endothelial cells. Unexpectedly, however, G10 did not induce expression of genes associated with the activity of NF-κB in fibroblasts even though such genes were induced in PBMCs and endothelial cells. Given the central role of NF-κB in generation of pro-inflammatory states that can lead to pathogenic consequences, especially under chronic circumstances, (reviewed in DiDonato J A et al, Immunol Rev 246, 379-400 (2012) and Tonatore L et al, Trends Cell Biol 22, 557-566 (2012); both of which are incorporated by reference herein), it is perhaps desirable that the activity of G10 is more transcriptionally focused to IRF3-dependent responses in certain cell types. It is also interesting that G10 induces type I IFN synthesis in the absence of detectable NF-κB activity given the reported requirement of the transcription factor for this process (Bartlett N W et al, EMBO Mol Med 4, 1244-1260 (2012); Falvo J V et al, Mol Cell Biol 20, 4814-4825 (2000); Wang J et al, J Immunol 185, 1720-1729 (2010); and Panne D et al, Cell 129, 1111-1123 (2007); all of which are incorporated by reference herein). Activation of noncanonical NF-κB subunits may play a role in this case. Undertaking a more thorough molecular investigation of NF-κB subunit activation (e.g. nuclear localization, phosphorylation, DNA binding) will be required to understand this with greater clarity. Importantly, G10 induced the phosphorylation of IRF3 and the protein's deletion led to elimination of reporter gene transcription as well as the compound's anti-Alphaviral activity. As such the innate biological effects of G10 examined here require IRF3-driven gene expression.

Deletion of the adaptor molecule IPS-1/MAVS did not eliminate G10-induced IRF3 phosphorylation or affect the molecule's antiviral effect (FIG. 5). Furthermore, G10-associated transcription of IRF3-dependent genes was also intact in the absence of IPS-1 (FIG. 6). In light of these results, it is intriguing that G10-induced IFN secretion was diminished following IPS-1 deletion (FIG. 7). This result is consistent with data showing interaction between IPS-1 and STING during RIG-I-mediated stimulation although whether STING strictly requires this interaction for full signaling has not been shown. Nevertheless, IPS-1 does not appear to play a substantial role in G10-mediated anti-Alphaviral activity and thus upstream IPS-1-dependent PRRs such as RIG-I and MDA5 are unlikely to be engaged by G10 or relevant for these effects.

Our results clearly establish an essential role for the signaling molecule STING. Deletion of the STING protein resulted in complete inactivation of G10-mediated IRF3 phosphorylation, IRF3-dependent transcription, IFN secretion, and antiviral activity (FIGS. 6 and 7). These results plainly signify that STING-dependent function(s) are necessary for the innate phenotypic response elicited by G10. Whether G10 represents a directly binding and activating synthetic ligand of human STING (as are DMXAA and CMA for mouse STING) was examined using thermal shift assays of purified protein. These revealed no increase in the thermal stability of STING-CTD in the presence of G10 as was seen for 2′3′-cGAMP (Supplemental FIG. 3). If the molecule bound directly to STING-CTD one would expect a net increase in the melting temperature of the protein dimers as observed when bona fide ligands such as 2′3′-cGAMP is co-incubated. Additionally, the inability of G10 to induce IRF3-dependent transcription in THP-1 cells (FIG. 15) is also not consistent with the molecule behaving as a direct ligand since these cells express biologically functional STING, as described in numerous studies. The identity of the protein or factor engaged by G10 that ultimately stimulates the STING-dependent response is currently under investigation. IRF3-activating, STING-dependent sensors such as IFI16, DDX41, and cGAS are also present and functional in THP-1 cells (Horan K A et al, J Immunol 190, 2311-2319 (2013); incorporated by reference herein). As such, it is probable that G10 either engages an alternative STING-dependent PRR such as ZBP1/DAI or IFI203 or an as yet uncharacterized STING-dependent PRR. ZBP1/DAI is a particularly attractive target since was previously shown it to be expressed and biologically active in THF cells.

Given that G10 stimulates innate cellular effects that require STING, it was decided to compare the dose dependence of these effects to 2′3′-cGAMP, an established STING ligand. Our results indicate that while G10 may trigger earlier IRF3 phosphorylation than 2′3′-cGAMP, perhaps due to its smaller size and cell permeability, it triggers levels of IRF3-dependent gene expression with overall less potency than 2′3′-cGAMP (FIG. 9). More precisely, 2′3′-cGAMP induces higher levels of IRF3- and IFN-dependent mRNA expression at lower concentrations than G10. It would be interesting to establish whether these dissimilarities are causally linked to differences in the molecules' cellular targets, especially whether their proximity in the signaling cascade to IRF3-directed kinases is important. Alternatively, differences in physico-chemical properties between the molecules and how those relate to solubility and permeability may also impact stimulatory potency (Lipinski C A et al, Adv Drug Deliv Rev 46, 3-26 (2001); incorporated by reference herein).

G10 induces synthesis and secretion of bioactive type I and III IFNs and generates an antiviral state in fibroblast cells positive for STING, IRF3, and STAT1 proteins. Based on these results our model for the elicitation of anti-Alphaviral activity by G10 first involves STING-dependent induction of IRF3 followed by IRF3-mediated synthesis and secretion of type I and III IFNs and subsequent IFN-stimulated, STAT1-dependent expression of antiviral effectors. Detection of STAT1-independent ISG expression in response to IFN exposure and IFN-inducing stimuli was unexpected but not unprecedented and has been reported in multiple studies (Hahm B et al, Immunity 22, 247-257 (2005) and Ousman S S et al, J Virol 79, 7514-7527 (2005); both of which are incorporated by reference herein. Blaszcyk and colleagues attribute this to IFN-induced transcriptional complexes composed of IRF9 and STAT2 homodimers although homo- and heterodimers of other Jak/Tyk2-phosphorylated STAT proteins may also play roles (reviewed in Brierly M M and Fish E N, Interferon Cytokine Res 25, 733-744 (2005). Interestingly, IFNβ was able to stimulate some antiviral activity in cells lacking STAT1 and to a degree that varied between viruses with SINV replication being undetectable. The full assortment of STAT1-independent ISGs expressed cannot be inferred from two proteins (Mx2 and ISG56) and as such the differential susceptibilities of CHIKV, VEEV, and SINV to ISG-encoded proteins in general cannot be known based on these results. Yet it is clear that SINV is highly sensitive to STAT1-independent ISGs relative to the other Alphaviruses. Intriguingly, however, while other IRF3-activating, IFN-inducing stimuli were capable of triggering expression of Mx2 and ISG56 in the absence of STAT1, G10 was not. This likely explains the reliance on STAT1 of G10-mediated anti-Alphaviral activity. Why this disparity in STAT1-dependence occurs between SeV, UV-CMV, and G10 is not clear. It is possible that each stimulus triggers the secretion of unique signatures of type I and type III IFN subtypes that subsequently elicit distinct gene expression patterns (Hilkens C M U et al, J Immunol 171, 5255-5263 (2003) and Moll H P et al, Cytokine 53, 52-59 (2011); both of which are incorporated by reference herein). Elucidation of the importance of the various IFN proteins in G10's antiviral effects will require more detailed examination, for instance by comparative transcriptomics, by using subtype-specific neutralizing antibodies or reverse genetics via gene editing.

While the majority of our investigation employed fibroblast cells, it is evident that G10 elicited innate immune activation in primary human cells such as PBMC's (FIG. 10) and umbilical endothelial cells (FIG. 16). Unexpectedly, however, induction of NF-κB-dependent transcription by G10 was observed in primary cells but not fibroblasts. Moreover, no G10-induced IRF3-dependent activity was detected in THP-1 cells (Supplemental FIG. 4). These disparities may be related to differences in cell type-specific expression of PRRs or innate signaling molecules, especially between stromal versus myeloid-derived cells (Arnold-Schrauf C et al, Eur J Immunol 45, 32-39 (2015); incorporated by reference herein) and between transformed and untransformed cells (Heiber J F and Barber G N, Methods Mol Biol 797 217-238 (2012); and references therein incorporated by reference herein). Understanding the biological bases for these divergent effects will require additional experimentation. However, demonstrating efficacy of G10 on primary human cells is obviously crucial to assessing the therapeutic potential of the compound.

Unfortunately G10 was unable to stimulate similar activation in murine cells. As such, evaluating the in vivo efficacy of G10 using a well-established mouse model of Alphavirus (CHIKV) infection was not directly practical. Yet IFN-inducing STING agonists (e.g. DMXAA, CMA) have been described that are murine specific. It was therefore examined whether DMXAA triggers IRF3 activation and IRF3-dependent gene induction in a manner comparable to G10. While comparisons of absolute responses are complicated by the fact that different species, cell types, and reagents are employed, DMXAA does trigger rapid IRF3 phosphorylation and dose-dependent IFNβ and ISG transcription in mouse cells (FIG. 11) as does G10 in human cells. In light of this the mouse model of acute CHIKV infection was used to ask whether activation of the STING pathway is feasible as an in vivo anti-Alphaviral strategy. DMXAA clearly blocks viremia but that this is related to the timing of administration with early (3 h pre- or 6 h post-infection) being more effective than late (24 h post-infection) treatment. It is probable that these kinetics correlate with the appearance of CHIKV-encoded IFN/ISG evasion phenotypes and as such STING-dependent antiviral efficacy diminishes with time post infection. However, whether STING activation represents an effective approach for diminishing persistent (e.g. >6 weeks) CHIKV infection (Hoarau J J et al, J Immunol 184, 5914-5927 (2010); incorporated by reference herein) is an enticing possibility that warrants examination since this could lead to alleviation of chronic virus-associated arthralgia and is currently being examined in our laboratory.

In summary a synthetic small molecule capable of inducing expression of type I and III IFNs as well as IFN-dependent antiviral effector genes was identified. Using a reverse genetics approach based on CRISPR/Cas9-mediated genome editing to identify cellular targets of the molecule it was shown that this effect requires STING, IRF3, and STAT1 proteins. These molecules are likewise essential to the ability of G10 to elicit a cellular state refractory to replication of Alphavirus species. Furthermore, given the pivotal role of STING it was also shown that pharmacologic activation of the molecule represents an effective anti-Alphaviral strategy in vivo. Given the demonstrated role of STING pathway stimulation in numerous immunological processes, it is being pursued as a therapeutic target for many diseases. Our work demonstrates the feasibility of identifying molecules that activate STING-dependent signaling and yield therapeutic outcomes as well as a strategy for characterizing cellular effects and essential modulatory proteins via genome editing.

### Example 12—Reagents and Antibodies

Dimenthyl sulfoxide (DMSO) was obtained from Thermo-Fisher. Puromycin was obtained from Clontech and used at 3 μg/mL in cell culture medium. Lipopolysaccharide (LPS) and polybrene were obtained from Sigma-Aldrich. Human recombinant IFNβ and tumor necrosis factor α (TNFα) were obtained from PBL. ONE-Glo cell lysis/luciferin reagent was obtained from Promega. Lucia luciferin reagent was obtained from Invivogen. Lipofectamine LTX was obtained from Life Technologies. Poly(I:C) was obtained from Amersham (27-4729). 2′3′-cGAMP and ppp-dsRNA were purchased from Invivogen (tlrl-cga23 and tlrl-3prna, respectively). Unless otherwise indicated cells were exposed to ppp-dsRNA at 12.5 μg/mL based on a dose response of innate immune activity performed on THF cells. Stocks of G10 were purchased from ChemDiv. DMXAA was purchased from ApexBio. Antibodies used against the following antigens are indicated in parentheses: GAPDH (Santa Cruz SC-51906); STAT1 (Santa Cruz SC-346) IRF3 (Santa Cruz SC-9082); human S386 phospho-IRF3 (Epitomics 2562-1); mouse S396 phospho-IRF3 (cell Signaling 4947); STING (Cell Signaling 3337); IPS-1 (Bethyl A300-782A); IFIT1/ISG56 (Thermo Fisher PA3 848); and Mx2 (Sigma HPA030235).

### Example 13—Cell and Virus Culture

Human foreskin fibroblasts originally obtained from the American Type Culture Collection were stably transduced with constitutively expressed human telomerase reverse transcriptase and the IRF3/IFN-responsive pGreenFire-ISRE lentivector and were maintained in DMEM containing 10% fetal calf serum (FCS) and antibiotics. Vero, BHK-21, and C6/36 cells were obtained from Alec Hirsch (Oregon Health and Science University) and were grown as described. RAW264.7 cells were obtained from Jay Nelson (Oregon Health and Science University) and transduced with a lentivector that contains firefly luciferase under the control of the type I IFN responsive element obtained from SA Biosciences. THP1-ISG Lucia cells were obtained from Invivogen and maintained in RPMI containing 10% FCS and antibiotics. These cells were differentiated in 100 nM phorbol 12-myristate 13-acetate (PMA) for 24 h before stimulation. Human peripheral blood mononuclear cells were obtained from StemCell Technologies and maintained in RPMI containing 10% FCS and antibiotics. Human umbilical microvascular endothelial cells were obtained from Patrizia Caposio (Oregon Health and Science University) and maintained as described in Botto S et al, Blood 117, 352-361 (2011); incorporated by reference herein. All cells were grown at 37° C. and 5% CO2. Sendai virus (SeV) was obtained from Charles River Laboratories and used at 16 HA units/mL. Cytomegalovirus was grown, titered, UV-inactivated, and exposed to cells as described previously (DeFilippis V R et al, J Virol 84, 8913-8925 (2010) and DeFilippis V R et al, J Virol, 80, 1032-1037 (2006); both of which are incorporated by reference herein. West Nile Virus (WNV) was obtained from Alec Hirsch (Oregon Health and Science University) and used as described in Hirsch A J et al, J Virol 79, 11943-11951 (2005); incorporated by reference herein. Vaccinia Virus (VACV) strain Western Reserve was obtained from Klaus Frith (Oregon Health and Science University) and used as described in Alzhanova D et al, Cell Host Microbe 6, 433-445 (2009); incorporated by reference herein. Sindbis virus (SINV) strain Ar-339 was obtained from ATCC. Venezeulan encephalitis virus (VEEV) strain TC83 and Chikungunya virus (CHIKV) strain MH56 were obtained from Michael Diamond (Washington University). CHIKV was derived from an infectious clone as follows. RNA was transcribed from the linearized clone using the T7 mMessage mMachine kit (Ambion) and transfected using Lipofectamine LTX into BHK-21 cells. Resultant virus was propagated in C6/36-insect cells for 48 h to produce high titer viral stocks after pelleting through a 20% sucrose cushion by ultracentrifugation (22,000 rpm, 825206 g for 1.5 hrs). In all cases infectious virus was quantified by serial dilution plaque assays on Vero cells with a carboxymethylcellulose overlay. Unless otherwise indicated experimental infections were carried out in triplicate using a multiplicity of infection (MOI) of 1 plaque forming unit (PFU) per cell. Cell viability was examined by quantitating ATP using the Cell Titer GLO assay according to the manufacturer's instructions (Promega).

### Example 14—Immunoblotting

Sodium dodecyl sulfate-polyacrylamide gel electrophoresis (SDS PAGE) immunoblots were performed as follows. After trypsinization and cell pelleting at 2,000×g for 10 min. whole-cell lysates were harvested in 2% SDS lysis buffer (50 mM Tris-HCl, 20% glycerol). Lysates were electrophoresed in 8% polyacrylamide gels and transferred onto polyvinylidene difluoride membranes (Millipore) using semidry transfer at 400 mA for 1 h. The blots were blocked at room temperature for 2 h or overnight using 10% nonfat milk in 1×PBS containing 0.1% Tween 20. The blots were exposed to primary antibody in 5% nonfat milk in 1×PBS containing 0.1% Tween 20 for 18 h at 4° C. The blots were then washed in 1×PBS containing 0.1% Tween 20 for 20, 15, and 5 min, followed by deionized water for 5 min. A 1 h exposure to horseradish peroxidase conjugated secondary antibodies and subsequent washes were performed as described for the primary antibodies. The antibody was visualized using enhanced chemiluminescence (Pierce).

### Example 15—RNA Isolation and Semiquantitative Reverse Transcription-PCR (RT-PCR)

Total RNA was isolated from cells and DNased using a DNA Free RNA Isolation kit according to the manufacturer's protocol (Zymo Research) and quantified by UV spectrometry. Single-stranded cDNA for use as a PCR template was made from total RNA using random hexamers to prime first-strand synthesis by Superscript III reverse transcriptase (Life Technologies) as described in the manufacturer's protocol. Comparison of mRNA expression between samples (e.g., treated versus untreated) was performed using semiquantitative real-time RT-PCR (qPCR) with the Applied Biosystems sequence detection system according to the MCT method (Livak K J and Schmittgen T D 25, 402-408 (2001); incorporated by reference herein). For IFNβ, IFNλ1, and mouse and human GAPDH (housekeeping gene) pre-validated PrimeTime FAM qPCR primer/probe sets obtained from IDT were used. For all other genes Maxima SYBR Green qPCR master mix (Thermo Fisher) was used. Primers for human ISG15, ISG56, ISG54, and Viperin were described in DeFilippis V and Frueh K, J Virol 79, 6419-6431 (2005); incorporated by reference herein).

Other human primer sequences were as follows:

| | |
|---|---|
| Mx2-For, | 5′-ACTTCAGTTCAGAATGGAG-3′; |
| Mx2-Rev, | 5′-TATTCTGTGAAGGCGTCC-3′; |
| OAS-For, | 5′-CAGCGCCCCACCAAGCTCAA-3′; |
| OAS Rev, | 5′-TGCTCCCTCGCTCCCAAGCA-3′; |
| IL8-For, | 5′-GACTTCCAAGCTGGCCGT-3′; |
| IL8-Rev, | 5′-GAATTCTCAGCCCTCTTCA-3′; |
| IL1β-For, | 5′-AACAGGCTGCTCTGGGATTCTCTT-3′; |
| IL1β-Rev, | 5′-TGAAGGGAAAGAAGGTGCTCAGGT-3′; |
| MIP1α-For, | 5′-GCTGCCCTTGCTGTCCTCCTC-3′; |
| MIP1α-Rev, | 5′-GGTCAGCACAGACCTGCCGG-3′. |

Mouse primers were as follows:

| | |
|---|---|
| IFIT2/ISG54-For, | 5′-TCCAGCCCCTACAGGATTGA-3′; |
| IFIT2/ISG54-Rev, | 5′-TTCGGGTCCTTTTCCAGAGC-3′; |
| IFNβ-For, | 5′-CTGGAGCAGCTGAATGGAAAG-3′; |
| IFNβ-Rev, | 5′-CTTCTCCGTCATCTCCATAGGG-3′; |
| Viperin-For, | 5′-AGCAGGTGTGTGCCTATCAC-3′; |
| Viperin-Rev, | 5′-TCAGCCAGCAGAACAGGATG-3. |

### Example 16—Lentivector Transduction and CRISPR/Cas9-Mediated Genome Editing

NF-κB responsive luciferase reporter cells were made using a commercially available replication incompetent lentivirus (Qiagen). Telomerized human fibroblasts were exposed to virus inoculum in the presence of DMEM plus 5 μg/mL polybrene and rocked at 37° C. for 8 h. At two days post inoculation cells were exposed to 3 μg/mL puromycin. After cells were fully resistant to puromycin they were verified for responsiveness to NF-κB-inducing stimuli (e.g. TNFα, SeV, LPS). Genome editing using lentivector-mediated delivery of CRISPR/Cas9 components was performed generally as described previously [56]. Briefly, 20 nt guide RNA (gRNA) sequences targeting protein-coding regions were inserted into the lentiCRISPRv2 vector (AddGene #52961). These sequences are as follows.

| | |
|---|---|
| IRF3: | GAGGTGACAGCCTTCTACCG; |
| IPS-1: | AGTACTTCATTGCGGCACTG; |
| STAT1: | AGAACACGAGACCAATGGTG; |
| STING: | CCCGTGTCCCAGGGGTCACG. |

Lentivirus was made by transfecting specific lentiCRISPRv2 plasmid along with packaging (psPAX2; AddGene #12260) and VSV-G pseudotyping (pMD2.G; Addgene #12259) plasmids into Lenti-X 293T cells (Clontech) using Lipofectamine-LTX (Life Technologies). Media was harvested at 48 h and 72 h post transfection, centrifuged (3,000×g for 10 min.) and filtered through a 0.45-μm-pore-size filter to remove cell debris. Subconfluent target cells were exposed to lentivirus for 8 h in the presence of 5 μg/mL polybrene. After the cells reached confluence they were split into DMEM plus 10% FCS containing 3 μg/mL puromycin. Transduced cells were passaged in the presence of puromycin for 7-10 days before protein knockout was examined by immunoblot. Cells were next serially diluted twice in 96 well plates to obtain oligoclonal lines purified for gene deletion. Protein knockout was additionally verified functionally by measuring phenotypic responsiveness to relevant stimuli as discussed below.

### Example 17—Luciferase Reporter Assays

Confluent reporter cells were plated at 20,000 (THF-ISRE) or 100,000 (THP1-ISG-Lucia) cells per well in a white 96 well plate 24 h before stimulation. Treatments were performed in quadruplicate in 50 μL DMEM plus 2% FCS for 7 h unless otherwise indicated. One-GLO lysis/luciferin reagent (Promega) was added at 1:1 to each well and luminescence measured on a Synergy plate reader (BioTek).

### Example 18—STING Protein Purification and Thermal Shift Assays

Coding sequences for human STING-C-terminal domain (CTD; AA 137-379) and mouse STING-CTD (AA 137-378) were cloned into pRSET-B vector (Invitrogen) and contained a 6×HIS tag for protein expression in E. Coli strain BL21 (DE3)pLysS (Promega). Sequences were verified before transforming bacteria, which were then grown in LB media 813 at 37° C. until the OD600 reached 0.8. Protein expression was induced with 1 mM IPTG at 16° C. for 18 h. After induction, the culture was centrifuged and the pellet resuspended in 50 mM NaH2PO4, 150 mM NaCl (pH 7.5) and 10% glycerol after which the cells were lysed by sonication. The recombinant soluble STING-CTD was purified by nickel-affinity chromatography (Clontech laboratories) after which it was further purified by gel-filtration chromatography using a HiPrep 16/60 Sephacryl S-100 HR column (GE Healthcare Life Sciences). Protein was eluted in 50 mM NaH2PO4, 150 mM Nacl (pH 7.5) and the eluted fractions containing STING-CTD concentrated using an Amicon centrifugal filter (10 Kd molecular weight cut-off; Millipore). Aliquots of concentrated STING-CTD were immediately stored at −80° C. For thermal shift assay, 1 μg of recombinant human or mouse STING-CTD was used combined with various concentrations of G10, 2′3′-cGAMP, or DMXAA along SYPRO Orange dye (1:1000 dilution) in a 204 reaction (in triplicate). A StepOne Plus Real-time PCR system was used to acquire fluorescence. The samples were subjected to a temperature gradient of 25 to 99° C. The melting curves were plotted and Tm values determined by fitting the curves to Boltzmann sigmoidal equation using the GraphPad Prism 6 software. Three independent experiments were performed.

### Example 19—In Vivo Administration of DMXAA and Viral Infection

C57Bl/6J mice (5-7 weeks of age, Jackson Laboratories) were housed in cage units in an animal BSL3 facility, fed ad libitum, and cared for under USDA guidelines for laboratory animals. 25 mg/kg DMXAA (or DMSO alone) was prepared in 50 μL DMSO and injected intraperitoneally. Mice were challenged with 1000 PFU CHIKV via footpad injection in 20 μL RPMI under isoflurane induced anesthesia. Animals were euthanized at 72 h post infection by isoflurane overdose. Blood was collected by cardiac puncture and serum viral loads titered on Vero cells in duplicate as described above.

### Example 20—Synthesis of G10 and Analogs

Methyl 3-oxo-3,4-dihydro-2H-benzo[b][1,4]thiazine-6-carboxylate (1) was prepared according to a literature procedure (Trifilenkov et al, J. Comb. Chem. 2006, 8, 469-479; incorporated by reference herein).

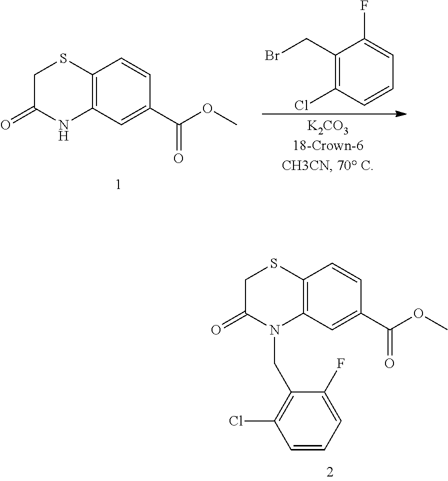 [SMILES: COC(=O)c1ccc2c(c1)NC(=O)CS2 ; Fc1cccc(Cl)c1CBr ; COC(=O)c1ccc2c(c1)N(Cc1c(F)cccc1Cl)C(=O)CS2]

Methyl 4-(2-chloro-6-fluorobenzyl)-3-oxo-3,4-dihydro-2H-benzo[b][1,4]thiazine-6-carboxylate (2). To a solution of compound 1 (670 mg, 3 mmol), potassium carbonate (6 mmol, 830 mg), and 18-Crown-6 (0.3 mmol, 79 mg) in 15 mL of dry acetonitrile was added 6-chloro-2-fluoro-benzylbromide (4.48 mol, 1.0 g). The resulting solution was heated to 70° C. for 24 hours and concentrated in vacuo. The residue was resuspended in chloroform and washed with water and brine, dried over anhydrous sodium sulfate and concentrated in vacuo to give a crude light-brown residue, which was used without further purification.

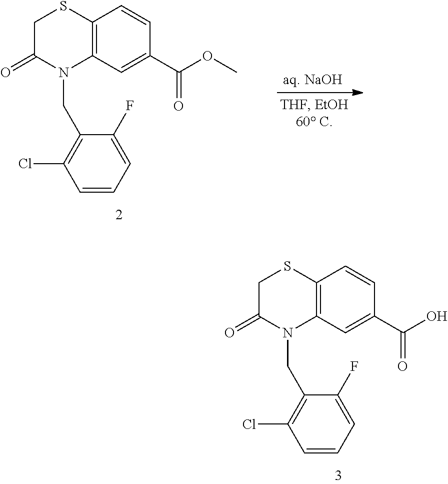 [SMILES: COC(=O)c1ccc2c(c1)N(Cc1c(F)cccc1Cl)C(=O)CS2 ; O=C(O)c1ccc2c(c1)N(Cc1c(F)cccc1Cl)C(=O)CS2]

4-(2-Chloro-6-fluorobenzyl)-3-oxo-3,4-dihydro-2H-benzo[b][1,4]thiazine-6-carboxylic acid (3). To a solution of crude compound 2 in 10 mL of THF and 10 mL of EtOH was added 15 mL of 2 M aqueous sodium hydroxide solution (6 mmol). The resulting solution was heated to 60° C. for 1 hour and concentrated in vacuo. The residue was resuspended in water and diethyl ether. The aqueous layer was separated (product) and acidified to pH ^{˜}2 with 1 M aqueous hydrochloric acid solution. Ethyl acetate was added, and the organic layer was washed with brine, dried over anhydrous sodium sulfate and concentrated in vacuo. Purification by column chromatography (0% to 10% methanol in dichloromethane) afforded the title compound 3 as a white solid (400 mg, 71% yield over two steps).

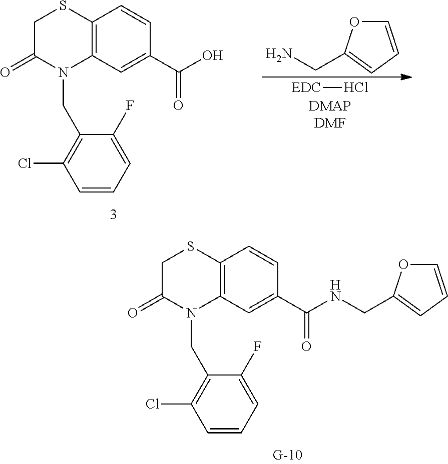

4-(2-Chloro-6-fluorobenzyl)-N-(furan-2-ylmethyl)-3-oxo-3,4-dihydro-2H-benzo[b][1,4]thiazine-6-carboxamide (G-10). To a solution of compound 3 (1.14 mmol, 400 mg), ethylcarbodiimide hydrochloride (1.71 mmol, 330 mg) and 4-dimethylaminopyridine (1.71 mmol, 210 mg) in 15 mL dry dimethylformamide was added furfuryl amine (1.71 mmol, 0.15 mL). The resulting solution was stirred at room temperature for 10 minutes and to it was added dry triethylamine (3.42 mmol, 0.48 mL). The reaction vessel was flushed with argon, and the reaction was allowed to stir at room temperature overnight. The reaction was diluted with ethyl acetate and washed with 0.5 M hydrochloric acid solution, water, saturated sodium bicarbonate solution, and brine. The organic layer was dried over anhydrous sodium sulfate and concentrated in vacuo. Purification by column chromatography (0% to 20% ethyl acetate in dichloromethane) afforded the title compound G-10 as a yellow solid (296 mg, 63% yield).

1. A method of identifying a test compound that is likely to act as an agonist of one or more proteins in the STING pathway, the method comprising:
contacting a first transfected human cell with the test compound, where the first transfected human cell comprises an expression vector, the expression vector comprising,
a first polynucleotide operably linked to a first promoter, the first polynucleotide encoding a human telomerase reverse transcriptase and where the first promoter is a constitutively active promoter
a second polynucleotide operably linked to a second promoter, the second polynucleotide encoding a bioluminescent protein or fluorescent protein and where the second promoter promotes the expression of the bioluminescent protein or fluorescent protein in the presence of interferon regulatory factor 3;
contacting a second transfected human cell with the test compound where the second transfected cell comprises the first polynucleotide operably linked to the first promoter and the second polynucleotide operably linked to the second promoter and where the first transfected human cell expresses STING and where the second transfected human cell does not express STING;
where expression of the bioluminescent or fluorescent protein in the presence of the test compound in the first transfected human cell that is greater than expression of the bioluminescent or fluorescent protein in the presence of the test compound in the presence of the test compound in the second transfected human cell is an indication that the test compound is likely to act as an agonist of one or more proteins in the STING pathway.

2. The method of claim 1, where the second promoter also promotes the expression of the bioluminescent protein or fluorescent protein in the presence of type I interferon.

3. The method of claim 1, where the second transfected human cell lacks expression of STING due to excising of the STING gene using CRISPR/Cas9.

4. The method of claim 1, where the bioluminescent or fluorescent protein comprises a luciferase.

5. The method of claim 1 further comprising contacting a third transfected human cell with the test compound, where the third transfected human cell comprises an expression vector, the expression vector comprising,
the first polynucleotide operably linked to the first promoter;
a third polynucleotide operably linked to a third promoter, the third polynucleotide encoding a bioluminescent or fluorescent protein and where the third promoter promotes the expression of the fluorescent protein in the presence of NF-κB;
where an expression level of the bioluminescent or fluorescent protein in the presence of the test compound in the third transfected human cell that is similar to or less than that of an expression level of the bioluminescent or fluorescent protein observed when contacting the third transfected human cell with a negative control compound is an indication that the test compound is likely not to induce NF-κB.

6. The method of claim 5 further comprising contacting the second transfected human cell with a positive control that induces NF-κB.

7. The method of claim 6 where the positive control comprises one or more of Sendai virus, tumor necrosis factor-α, or lipopolysaccharide.

8. The method of claim 1 where the test compound comprises a positive control compound.

9. The method of claim 8 where the positive control comprises a compound with the formula:

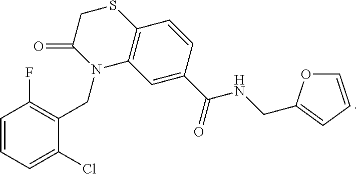 [SMILES: O=C(NCc1ccco1)c1ccc2c(c1)N(Cc1c(F)cccc1Cl)C(=O)CS2]

10. A compound with the formula: 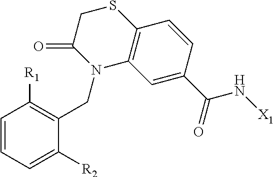
where X₂ is aryl or aryl substituted alkyl, and where R₁ and R₂ are independently H or halo.

11. The compound of claim 10 with the formula: 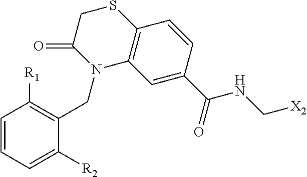
where X₂ is aryl.

12. The compound of claim 11 where X₂ is phenyl or furanyl.

13. The compound of claim 12 with a formula selected from

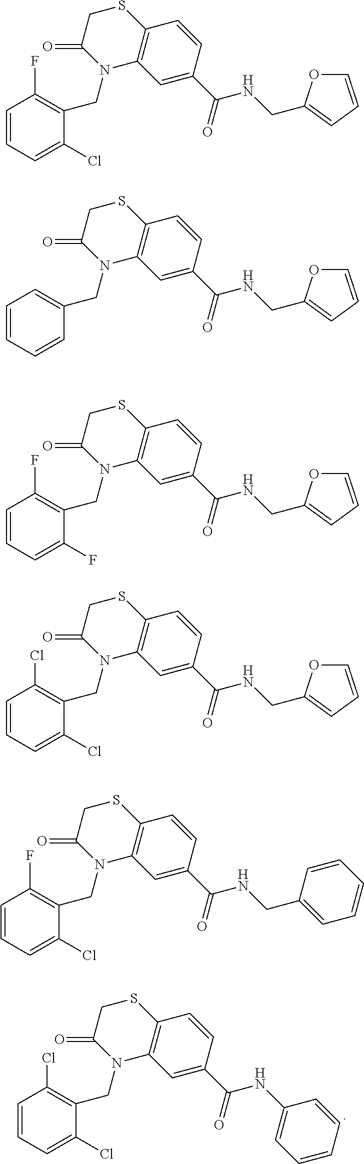

14. A pharmaceutical composition comprising an effective amount of the compound of claim 10 and a pharmaceutically acceptable carrier.

15. A method of inhibiting alphavirus replication in a subject, the method comprising:
administering the pharmaceutical composition of claim 14 to the subject, thereby inhibiting the alphavirus replication.

16. The method of claim 15 where the pharmaceutical composition comprises a structure with the formula

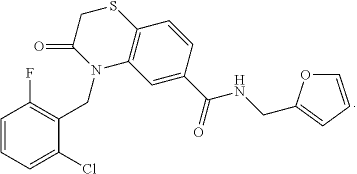 [SMILES: O=C(NCc1ccco1)c1ccc2c(c1)N(Cc1c(F)cccc1Cl)C(=O)CS2]

17. The method of claim 15 where the alphavirus is chikungunya virus.
